(19) United States
(12) Patent Application Publication
(10) Pub. No.: US 2022/0221119 A1
(43) Pub. Date: Jul. 14, 2022

(54) CANDLE SIMULATORS

(71) Applicant: Luminous Innovations, Inc., Portola Valley, CA (US)

(72) Inventors: John Jacobson, Portola Valley, CA (US); Michael Mandanis, Portola Valley, CA (US)

(21) Appl. No.: 17/574,440

(22) Filed: Jan. 12, 2022

Related U.S. Application Data
(60) Provisional application No. 63/136,521, filed on Jan. 12, 2021.

(51) Int. Cl. F21S 10/04; A61L 9/14

(52) U.S. Cl. CPC F21S 10/046; A61L 2209/134; A61L 2209/12; A61L 9/14

(57) ABSTRACT

Particular embodiments described herein include an apparatus for providing a simulated flame that includes a base housing, a flow generator contained within the base housing that is configured to generate a flow of atomized fluid, a lid positioned on top of the base housing, the lid defining a main opening through which the flow of atomized fluid is configured to be emitted, a chimney that is attached to a top surface of the lid and that, at least partially, surrounds the opening, the chimney extending upward from the top surface of the lid and being configured to focus, at least in part, the flow of atomized fluid into a channel of atomized fluid, and one or more light sources that are positioned near the opening to the lid that are configured to illuminate the channel of atomized fluid to provide the simulated flame.

### CROSS-REFERENCE TO RELATED APPLICATIONS

This application claims priority to U.S. Provisional Application No. 63/136,521, filed Jan. 12, 2021, the contents of which are incorporated herein by reference in their entirety.

### TECHNICAL FIELD

This document generally describes devices, systems, and methods related to candle simulators.

### BACKGROUND

Candles have traditionally included a wick that is lit to provide a flame that generates light. Wicks can be embedded in wax or other apparatus to hold the wick in place as it burns and emits light. Candles can be used for a variety of purposes, such as to illuminate dark environments (e.g., dark rooms) and/or to add an aesthetic appeal to a room or other setting. For example, a candle's flame can be ignited to provide aesthetic appeal within a room.

### SUMMARY

The document generally relates to candle simulators, which can include devices and apparatus that simulate a candle flame without a wick (or other ignitable object) and without an actual flame. The disclosed technology can provide for realistic flame simulation in a manner that can generate the same (or better) aesthetic appeal of an actual candle without the use of an actual flame, which can improve user safety by reducing the risk of candles accidentally igniting other objects, such as in the case of an actual candle being knocked over and/or being placed too close to other flammable objects.

The disclosed technology can provide flame simulations that are highly realistic without the fire-related risks posed by actual candles. Realistic flame simulations can be generated by the candle simulation disclosed in this document through a variety of features. For example, a candle simulator can include an atomizer to atomize a fluid (e.g., water) that can be illuminated by one or more light sources to simulate a flame. However, channeling a flow of atomized fluid (e.g., water vapor, mist) to appear as a realistic flame, including flickering with changing intensities of light and concentration, is not trivial. To provide realistic flames, the disclosed candle simulators can include a blower that is configured to generate a flow of the atomized fluid that is directed through an aperture and the light sources can be positioned in and/or around the aperture to illuminate the atomized fluid. The atomized fluid can be channeled into a simulated flame by a chimney structure at the aperture and/or by one or more smaller holes that are formed in one or more portions of the chimney structure. A transparent enclosure (e.g., glass enclosure, plastic enclosure) can be configured to surround the simulated flame and to extend at least a minimum distance from the base of the simulated flame. Additional and/or alternate features can be used to generate realistic simulated flames.

The disclosed candle simulators can include a variety of additional features that are designed to mitigate and/or solve other issues that may be introduced by the use of candle simulators. For example, although candle simulators may not pose fire risks like traditional candles, they can include a reservoir of fluid (e.g., water) that may be possible to spill if the candle simulator is tipped over. The disclosed simulators solve and alleviate these (and other) issues, for example, by providing a spill-proof fluid reservoir that is configured to mitigate and/or stop water from seeping out of the reservoir when the candle simulator is tipped over. Additionally, the disclosed candle simulators can include one or more openings in a top surface of the candle simulator that can be used to conveniently refill the water reservoir without having to tip the simulator on its side and/or otherwise disassemble the candle simulator to gain access to the reservoir. Such openings in the top surface of the candle simulator can be covered up by one or more decorative components, though, which can maintain aesthetics of candle simulators while providing for enhanced functionality and use.

The disclosed candle simulators can include component that generate aromas and/or scents, which can also simulate aromas and/or scents that are generated by actual candles. For example, the disclosed candle simulators can include components that permit for scented fluid to be atomized and emitted from the candle simulator as atomized scented fluid (e.g., scented vapor, scented mist). Such components can include, for example, an additional atomizer and fluid reservoir to retain and atomize scented fluid, an additional fluid reservoir to retain and dispense scented fluid into a combined atomizer to atomize scented fluid together with a primary fluid (e.g., water) for the simulated flame, and/or a combined reservoir for scented fluid and a primary fluid that feeds an atomizer. In the case of an additional atomizer, the atomized scented fluid may be combined with the atomized primary fluid (e.g., water vapor) and emitted from the same aperture, and/or it may be separately emitted from one or more different apertures in the candle simulator. Scented fluids may be filled in a variety of ways, such as through refilling from a supply of scented fluid, through the use of replaceable scent pods, which may contain a volume of scented fluid, concentrated material that can be combined with the main fluid to generate scented fluid (e.g., dissolved), and/or other components.

The disclosed candle simulators can additionally and/or alternatively include additional features that are not present with actual candles, such as components to generate sound (e.g., embedded speaker), components to generate additional sources of light beyond the simulated flame, components to permit for remote control/operation of the candle simulators, and/or components to permit for coordinated operation among multiple different candle simulators.

The disclosed candle simulators can additionally and/or alternatively be designed to permit for efficient and cost effective manufacturing through the use of several swappable components that allow for a wide variety of designs to be readily achieved without requiring a vast number of different manufacturing lines. For example, the candle simulators can include a common module that contains the atomizer(s), fluid reservoir(s), blower, and/or lighting devices, which can be inserted into a variety of differently shaped, sized, and/or patterned outer housings. These outer housings can additionally include different design elements, such as different chimney designs, different top surfaces from which the chimneys extend, different transparent shields, and/or other components that can be readily combined to great a vast number of different candle simulators.

Particular embodiments described herein can include an apparatus for providing a simulated flame, the apparatus including a base housing, a flow generator contained within the base housing that can generate a flow of atomized fluid, a lid positioned on top of the base housing, the lid defining a main opening through which the flow of atomized fluid can be emitted, a chimney that can be positioned adjacent the main opening, the chimney extending upwards and that can focus, at least in part, the flow of atomized fluid into a channel of atomized fluid, and one or more light sources that can be positioned near the main opening to the lid that can illuminate the channel of atomized fluid to provide the simulated flame.

Such an apparatus can optionally include one or more of the following features. For example, the chimney can be attached to a top surface of the lid and that, at least partially, surrounds the opening, the chimney extending upward from the top surface of the lid, in which the chimney can focus, at least in part, the flow of atomized fluid into the channel of atomized fluid. One or more sidewalls of the chimney can also define one or more apertures that can promote, at least in part, the formation of the channel of atomized fluid by the chimney. The one or more sidewalls can extend orthogonally from the top surface of the lid. The one or more sidewalls can include one or more curved surfaces that can extend from the top surface of the lid. The one or more sidewalls can include one or more planar surfaces that can extend from the top surface of the lid. Moreover, the one or more sidewalls can taper from their attachment to the top surface of the lid to a terminal point above the top surface.

The apparatus can also include a transparent lid that can extend upward from a top surface of the lid, the transparent lid at least partially enclosing a volume that can contain the simulated flame. The transparent lid can define a first opening that can mate with the lid and a second opening that can be open to an ambient environment. In some implementations, the chimney can be part of the base housing. The chimney can also extend through the main opening to the lid.

As another example, the apparatus can also include a cloud chamber embedded inside the base housing and fluidically connected to the main opening to the lid and the flow generator, a liquid chamber embedded inside the base housing and positioned beneath a portion of the cloud chamber, and a valve positioned inside the base housing to fluidically separate the cloud chamber from the liquid chamber. The valve can prevent liquid from flowing from the liquid chamber into the cloud chamber. The valve can be a one-way valve. The valve can be a silicone valve. Moreover, the apparatus can include a fan embedded inside the base housing, the fan being configured to circulate the flow of atomized fluid from the flow generator through the cloud chamber and out through the main opening to the lid to provide the simulated flame. A speed of the fan can be adjustable so as to change an appearance of the simulated flame. A higher fan speed can increase the flow of atomized fluid to provide a stronger simulated flame and a lower fan speed can decrease the flow of atomized fluid to provide a slower simulated flame.

Particular embodiments described herein include an apparatus for providing a simulated flame that includes a base housing, a flow generator contained within the base housing that is configured to generate a flow of atomized fluid, a lid positioned on top of the base housing, the lid defining a main opening through which the flow of atomized fluid is configured to be emitted, a chimney that is attached to a top surface of the lid and that, at least partially, surrounds the opening, the chimney extending upward from the top surface of the lid and being configured to focus, at least in part, the flow of atomized fluid into a channel of atomized fluid, and one or more light sources that are positioned near the opening to the lid that are configured to illuminate the channel of atomized fluid to provide the simulated flame.

Such an apparatus can optionally include one or more of the following features. One or more sidewalls of the chimney can define one or more apertures that are configured to promote, at least in part, the formation of the channel of atomized fluid by the chimney. The one or more sidewalls can extend orthogonally from the top surface of the lid. The one or more sidewalls can include one or more curved surfaces that extend from the top surface. The one or more sidewalls can include one or more planar surfaces that extend from the top surface. The one or more sidewalls can taper from their attachment to the top surface of the lid to a terminal point above the top surface. The apparatus can further include a transparent lid that extends upward from the top surface of the lid, the transparent lid at least partially enclosing a volume that is configured to contain the simulated flame. The transparent lid can define a first opening that is configured to mate with the lid and a second opening that is configured to be open to an ambient environment.

In some embodiments, the candle simulation designs depicted in one or more of the figures.

The devices, system, and techniques described herein may provide one or more of the following advantages. For example, a top portion of the candle simulator can have apertures for creating a more realistic flame. The apertures can be angled and configured in such a way that when mist is expelled through the apertures and a light source illuminates the mist from below, the mist can have a more realistic flame-like appearance.

As another example, the candle simulator can be configured to glass lids of different heights to accommodate for different flame heights or types. In other words, the simulator can be fitted into a variety of differently sized containers and lids. A lid with a bigger height can be used advantageous where the candle simulator emits a larger faux flame. A lid with a smaller height can be advantageous where the simulator emits a smaller faux flame. Either lid can be fitted or attached to the simulator to accommodate a user's desired preferences.

As yet another example, the disclosed technology can provide for ease of use in refilling a water cartridge or tank of the candle simulator. A user can pour water through an opening in the top of the simulator. The opening can include a funnel for funneling the water into the cartridge so that the water does not spill out of the simulator or onto other components (e.g., electrical components) of the simulator. The opening can also include a meter line visible from a top of the simulator such that the user can easily see when they are filling water up to a capacity of the water cartridge or tank.

As another example, the disclosed technology can provide for reducing spillage of water from the candle simulator. The encasing and sealed top of the simulator can prevent water from spilling out of the water cartridge if the simulator is tipped over or otherwise not on a flat surface. The sealed top can be made of silicone and have a double lip lid around a top portion of the simulator's housing. This configuration can seal the simulator such that water within the simulator (e.g., inside the water cartridge or tank) may not spill out.

The disclosed technology can also provide for improved safety since the simulator does not generate a real flame. The simulator generates a realistic looking flame that can also emit a fragrance or other desired aroma. Since the simulator does not generate a real flame, the simulator may not create a fire hazard or other safety concern when used in an indoor or other setting.

The details of one or more implementations are set forth in the accompanying drawings and the description below. Other features and advantages will be apparent from the description and drawings, and from the claims.

### BRIEF DESCRIPTION OF THE DRAWINGS

FIGS. 1A-B depict an example candle simulator with a metal lid attachment.

FIG. 1C depicts the metal lid attachment for the candle simulator of FIGS. 1A-B.

FIG. 1D depicts removing the metal lid attachment from the candle simulator of FIGS. 1A-C.

FIG. 2A depicts a candle simulator module that can be placed within different candle simulator housings.

FIG. 2B depicts an example candle simulator with a silicon lid attachment.

FIG. 2C depicts example configurations of the candle simulator with the metal lid attachment described herein.

FIGS. 3A-B depicts the silicon lid attachment and a metal plate chimney for the example candle simulator of FIG. 2B.

FIG. 3C depicts components of the candle simulator with the silicon lid attachment of FIG. 2B.

FIG. 3D depicts removing components of the candle simulator with the silicon lid attachment of FIG. 2B.

FIG. 3E depicts a bottom view of the silicon cap described herein.

FIG. 3F depicts attachment of the silicon cap to the candle simulator.

FIG. 3G depicts attachment of the metal lid attachment to the candle simulator.

FIG. 4 depicts an example candle simulator module of FIG. 2A.

FIG. 5A depicts a bottom view of the candle simulator described herein.

FIG. 5B depicts a bottom view of the candle simulator with cable management.

FIGS. 6A-B are schematic cutout side views of an example candle simulator.

FIG. 6C is a cutout side view of components of the candle simulator having the metal lid attachment.

FIG. 7 is a schematic cutout side view of the candle simulator having the metal lid attachment.

FIG. 8A is a top down view of the candle simulator having the silicon lid attachment.

FIG. 8B is a top down view of the candle simulator having the metal lid attachment.

FIG. 9 is an exploded top down view of the metal plate chimney and components of the candle simulator module.

FIGS. 10A-B depict the candle simulator module.

FIG. 10C depicts a top view of the candle simulator having the silicon lid attachment.

FIG. 11 depicts the candle simulator module when tipped at an angle.

FIGS. 12A-C depict an example candle simulator with an insertable fragrance bottle.

FIG. 13 is a schematic cutout side view of an example candle simulator.

FIG. 14 depicts a front view of an example candle simulator without a transparent lid.

Like reference symbols in the various drawings indicate like elements.

### DETAILED DESCRIPTION OF ILLUSTRATIVE EMBODIMENTS

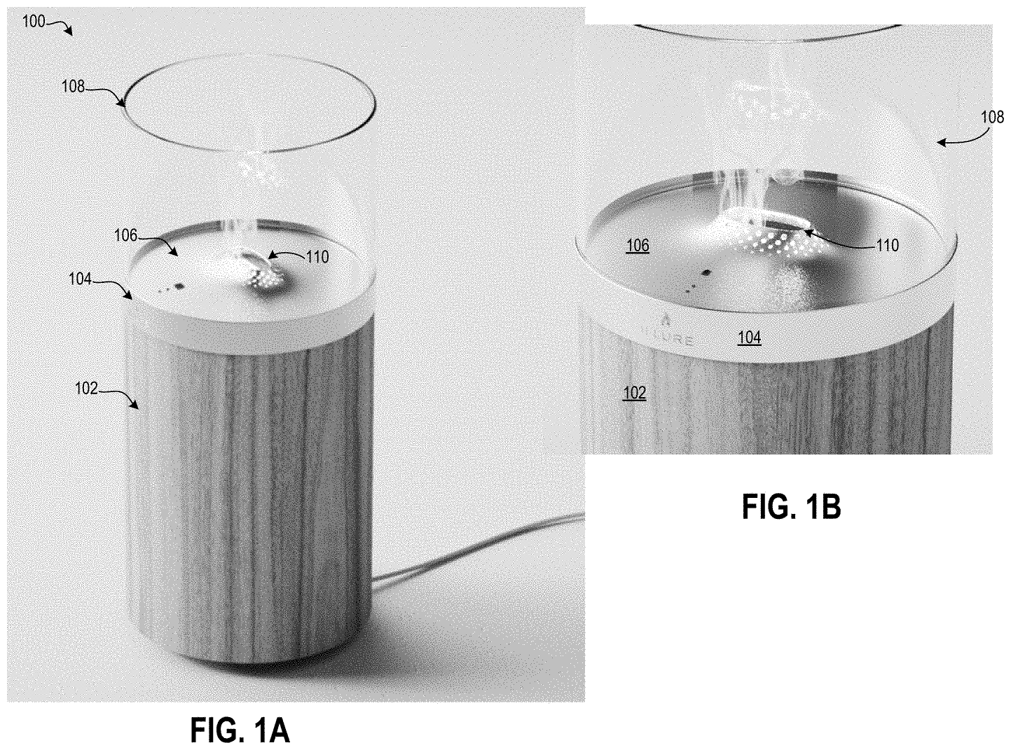

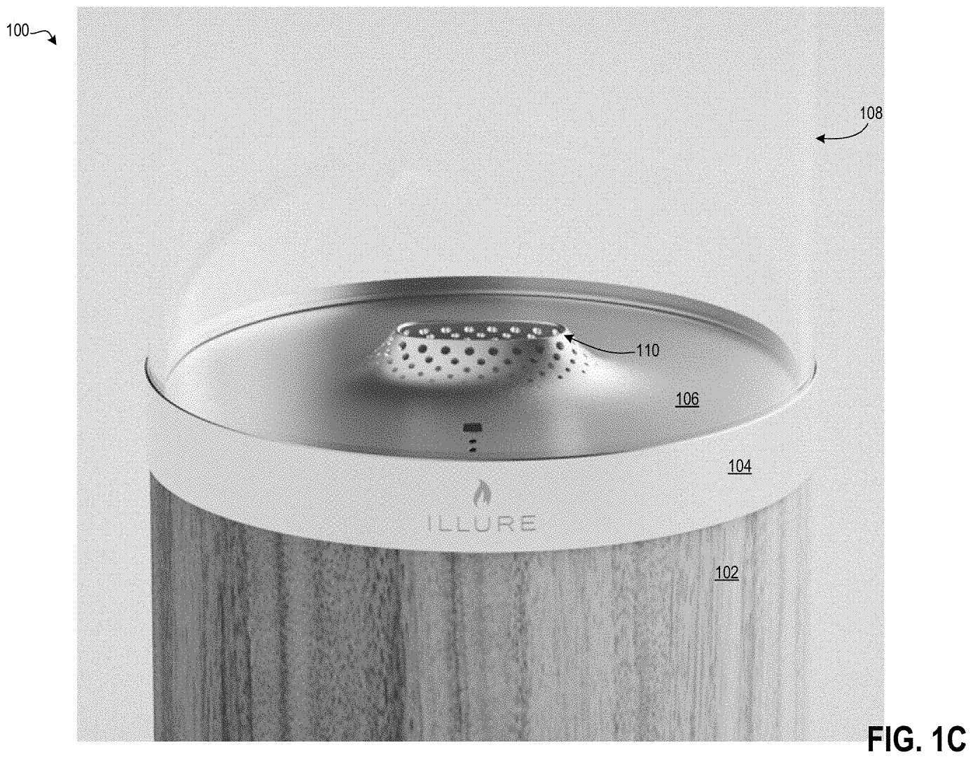

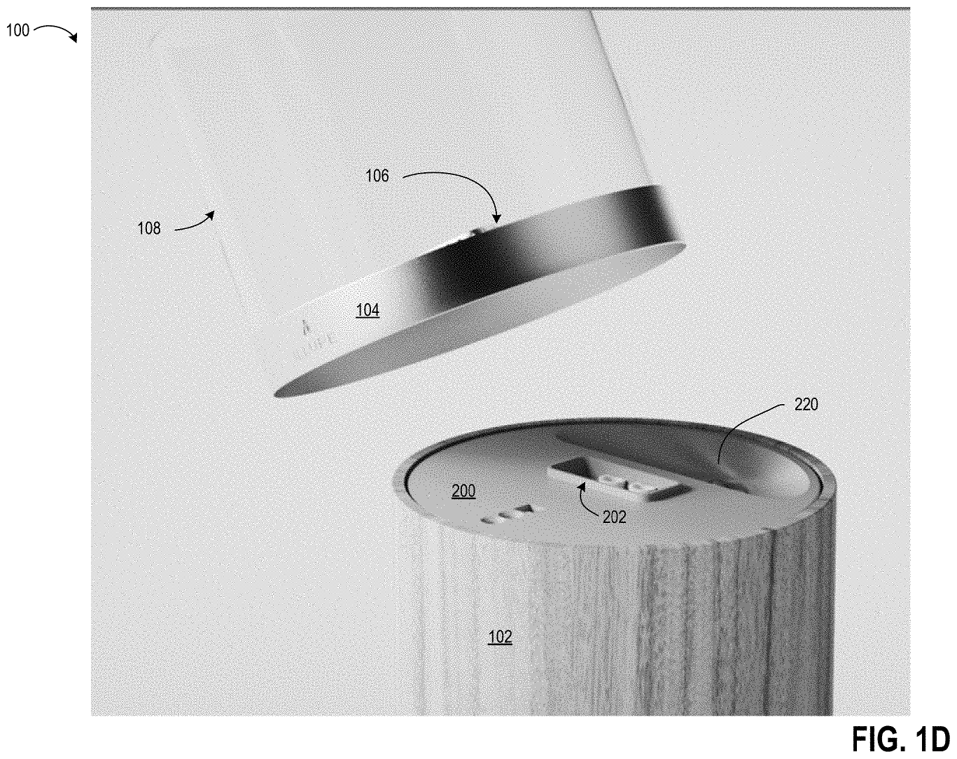

This document generally relates to candle simulators that generate realistic looking flames and, in some instances, includes a variety of additional features, such as components to emit aromas and/or other fragrances. Referring to FIGS. 1A-B, an example candle simulator 100 with a lid attachment 104 is depicted. FIG. 1C depicts the lid attachment 104 for the candle simulator 100 of FIGS. 1A-B. Referring to FIGS. 1A-C, the simulator 100 can include a sleeve 102, the lid attachment 104, a top surface 106, and a transparent lid 108. The sleeve 102 can be decorative and can come in any of a variety of different patterns, designs, shapes, and/or sizes, and can combined with the lid attachment 104 to provide a base for the candle simulator 100. One or more components to generate a realistic flame simulation can be contained within such a base, such as through the use of a cartridge including components to generate a flow of atomized and illuminated fluid. The sleeve 102 and the lid 104 can be provided in any of a variety of designs that can be wrapped around the cartridge, which can permit for a variety of different candle simulator designs to be achieved through different sleeve 102 and/or lid 104 combinations while using the same cartridge/internal components.

The top surface 106 can include a chimney 110 with a main aperture/opening through which flow of atomized fluid (e.g., water vapor) is emitted via a blower contained within the candle simulator 102. The atomized fluid can be illuminated by one or more lights that are positioned inside of or near the chimney 110, which can provide a simulated flame feature. The chimney 110 can include a variety of additional and smaller shaped apertures in its sidewall to promote the formation of a flow of atomized fluid that, when illuminated, provides a realistic flame simulation. The apertures in the sidewalls of the chimney 110 can, for example, promote a central column of atomized fluid to be projected through the opening/main aperture while minimizing pockets of lower pressure adjacent to the opening and near the top surface 106, which can avoid pressure-based short-cycling that would cause the centralized column of atomized fluid to spill over onto the surface. By being able to provide and maintain a centralized and focused column of atomized fluid above the main opening of the chimney 110 (through the use of the chimney 110 and its sidewall apertures, for example), the simulated flame can appear more realistic and can retain its realistic flame appearance for extended durations. The lid attachment 104, its top surface 106 and/or chimney 110 can be made from any of a variety of materials, such as metal materials, wood materials, silicon materials, plastic materials, and/or others. An example material can be aluminum, but any other lightweight metal material can be used. The components of the lid attachment 104 can be constructed from separate components and/or materials, as well. For example, the chimney 110 can be integrated into and/or attached to the surface 106.

The transparent lid 108 can be configured to be connected to (e.g., attach, rest on top of) the sleeve 102 and/or the lid attachment 104 so as to enclose (fully and/or partially) a volume around the simulated flame being emitted through the chimney 110. The transparent lid 108 can be any of a variety of shapes, such as a tube (e.g., straight tube, curved tube, tapered tube), a structure with one or more straight sides (e.g., box, cube, tube with one or more straight sides), irregular shapes, and/or others. The transparent lid 108 can be open at the bottom to receive the simulated flame and the sleeve 102/lid attachment 104. The transparent lid 108 can include one or more other openings to permit for the atomized fluid to be evacuated from the candle simulator and to permit for airflow to recirculate into the areas adjacent to the chimney 110 (to avoid pockets of low pressure). The transparent lid 108 may be configured to be spaced apart laterally from the chimney 110 so as to permit for airflow around the central channel of atomized fluid that is forming the simulated flame. The transparent lid 108 can be made of any of a variety of materials, such as glass (e.g., hurricane glass), plastic, and/or other at least semi-transparent materials. The lid 108 can also be in varying heights. A higher lid 108 can provide for a higher flame while a lower lid 108 can provide for a lower flame. A user can choose which size lid 108 to use to achieve a desired flame height. The sleeve 102 can come in different patterns, sizes, textures, and/or colors.

FIG. 1D depicts removing the lid attachment 104 from the candle simulator 100 of FIGS. 1A-C. The top surface 106, the transparent lid 108, and the lid attachment 104 can be sealed or attached together. As shown, the lid attachment 104 can be removed or detached from the sleeve 102 such that a simulator module 200 is accessible. The simulator module 200 can house one or more components that are used for generating the realistic-looking flame and/or emitting an aroma or other fragrance, as described further below. The simulator 200 can include openings 202 and 220. The opening 202 can be configured to mate with the chimney 110 such that the mist can flow from within the module 200 and through the chimney 110. The mist can be illuminated by lighting features through this flow path. The opening 220 can also be configured for receiving water to fill a water tank or cartridge within the module 200. A user can therefore remove the lid attachment 104 to access the water cartridge of the module 200 and fill the cartridge with water. The user can then replace the lid attachment 104, thereby sealing the water within the water cartridge to avoid from spilling.

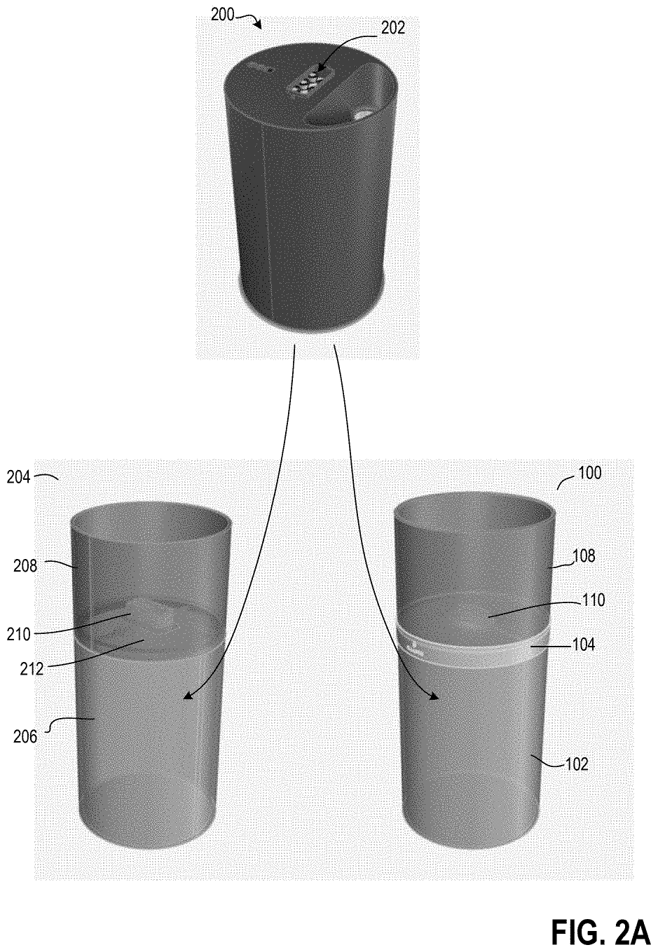

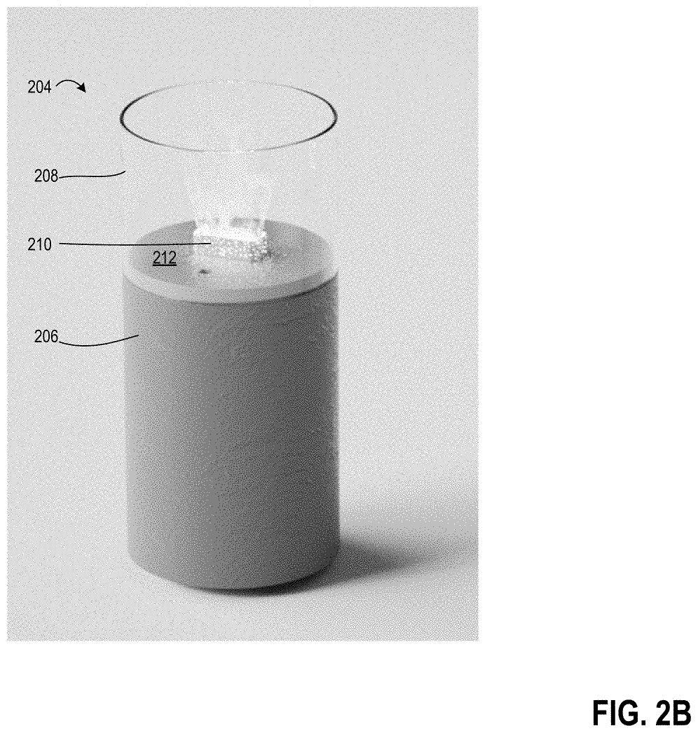

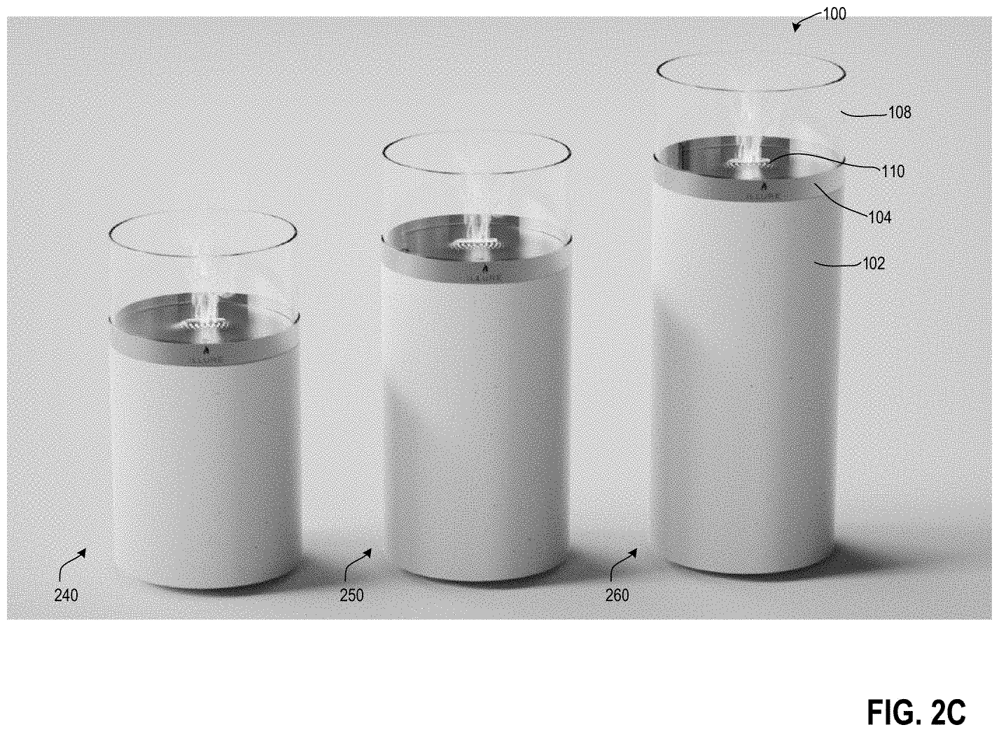

FIG. 2A depicts a candle simulator module 200 that can be placed within different candle simulator housings 204 and 100. The module 200 can fit into a silicon lid attachment housing 204. The module 200 can also fit into the simulator housing 100 having the lid attachment 104 described herein (e.g., refer to FIGS. 1A-D).

The housing 204 can include a glass lid 208 and a sleeve 206. The glass lid 208 and/or the sleeve 206 can be the same or similar to the transparent lid 108 and/or the sleeve 102 of the housing 100. Moreover, as depicted, the housing 204 can include a metal plate chimney 210 configured to a silicon cap 212. The glass lid 208 can also be configured to the silicon cap 212. When the silicon cap 212 is removed or otherwise detached from the sleeve 206, the glass lid 208 can also be removed so that the user can access components of the module 200 (e.g., fill the water cartridge of the module 200 with water). The opening 202 can also be aligned with the metal plate chimney 210. The metal plate chimney 210 can be removably connected to the silicon cap 212. For example, the chimney 210 can be screwed, bolted, or otherwise fastened to the silicon cap 212. The chimney 210 can optionally be replaced with other chimneys, which can provide for variation in flame type, size, and style.

FIG. 2B depicts an example candle simulator with the silicon lid attachment 204. The sleeve 206 can be a variety of different colors, styles, sizes, and/or textures, as depicted and described herein. As shown, a faux flame is emitted through the metal cap 210 that is fastened to the silicon cap 212. The glass lid 208 is a shorter height than other glass lids depicted and described herein, which can provide for a wider and/or shorter faux flame. Glass lids of one or more other sizes/heights can be configured to the simulator 204 to provide for the user's desired flame style and/or height.

FIG. 2C depicts example configurations of the candle simulator 100 with the lid attachment 104 described herein. As shown, the simulator 100 can come in a variety of sizes, such as a small 240, a medium 250, and a large 260. The simulator 100 can also have a variety of textures, patterns, and/or colors. Although not depicted, the candle simulator with the silicon lid attachment 204 can also come in similar sizes (e.g., small, medium, and large). In some implementations, the simulators 100 and/or 204 can also have different sized glass lids 108 and 208. For example, the simulator 100 can be in the medium size 250 with the transparent lid 108 having a longest height.

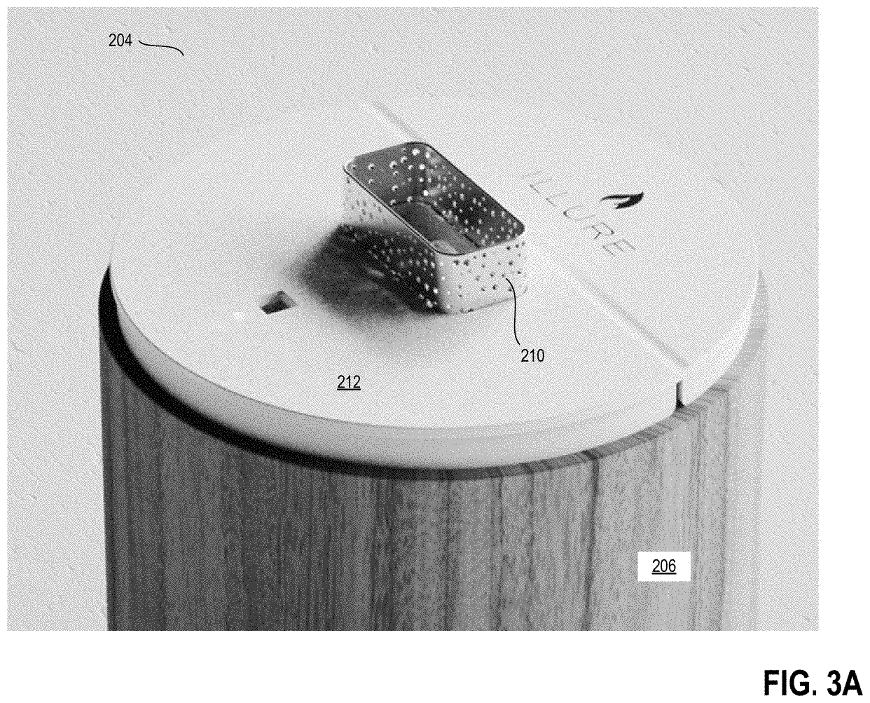

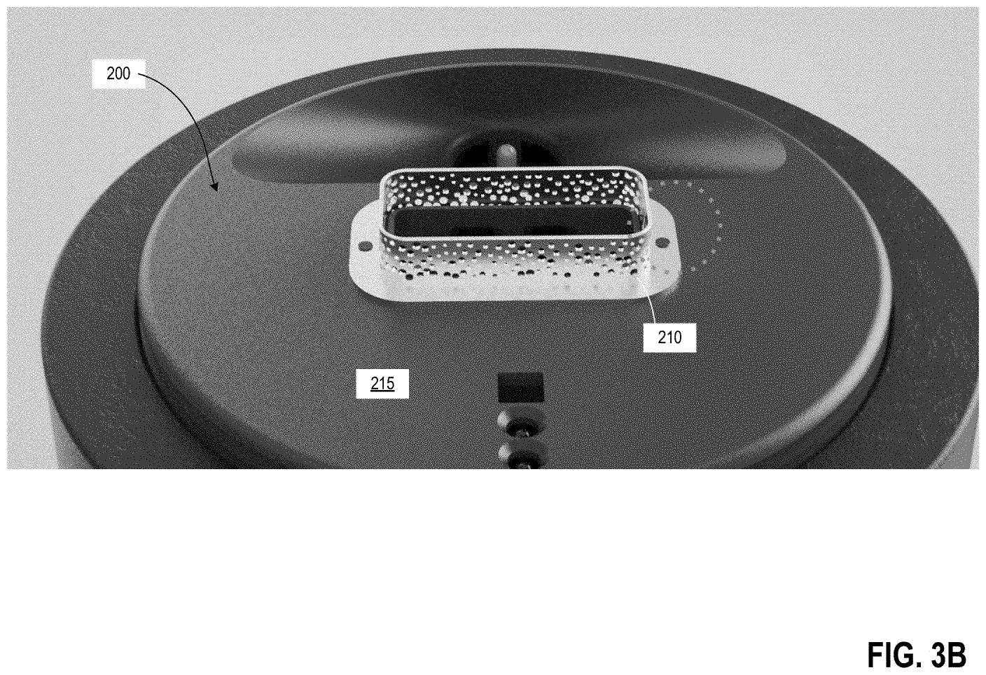

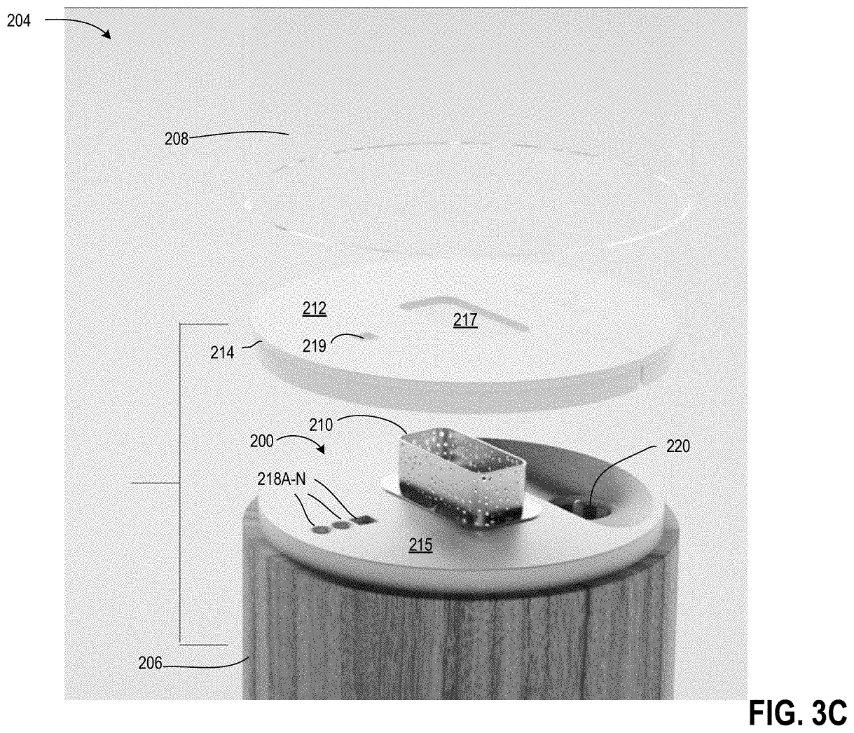

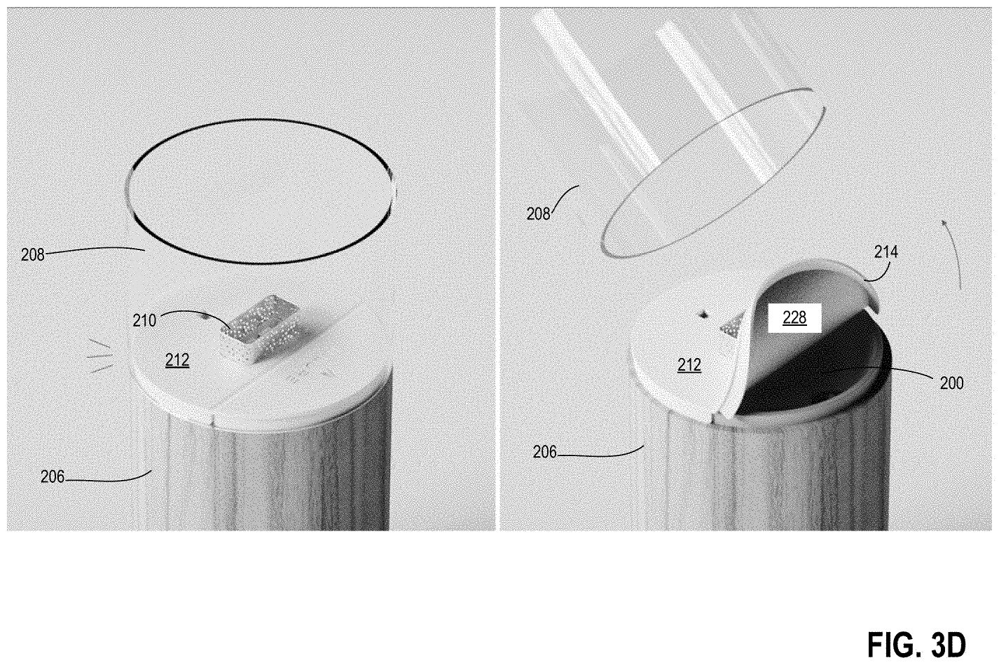

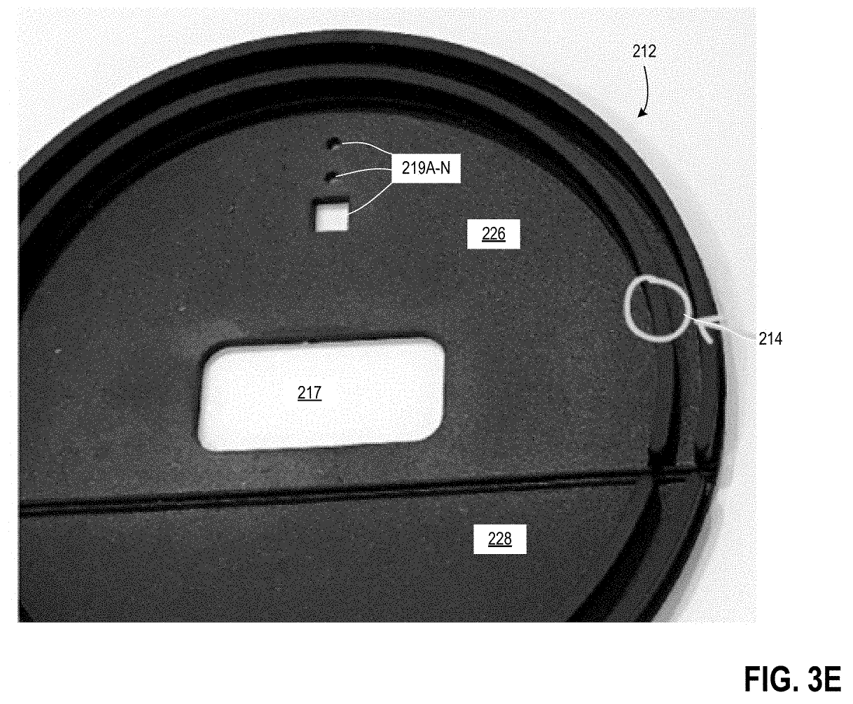

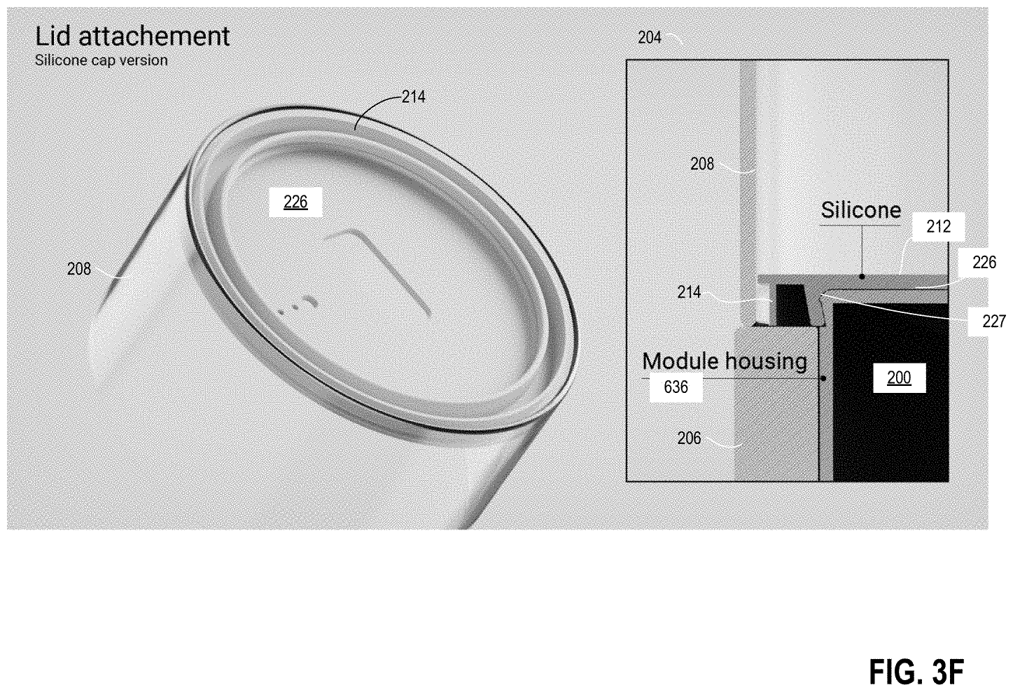

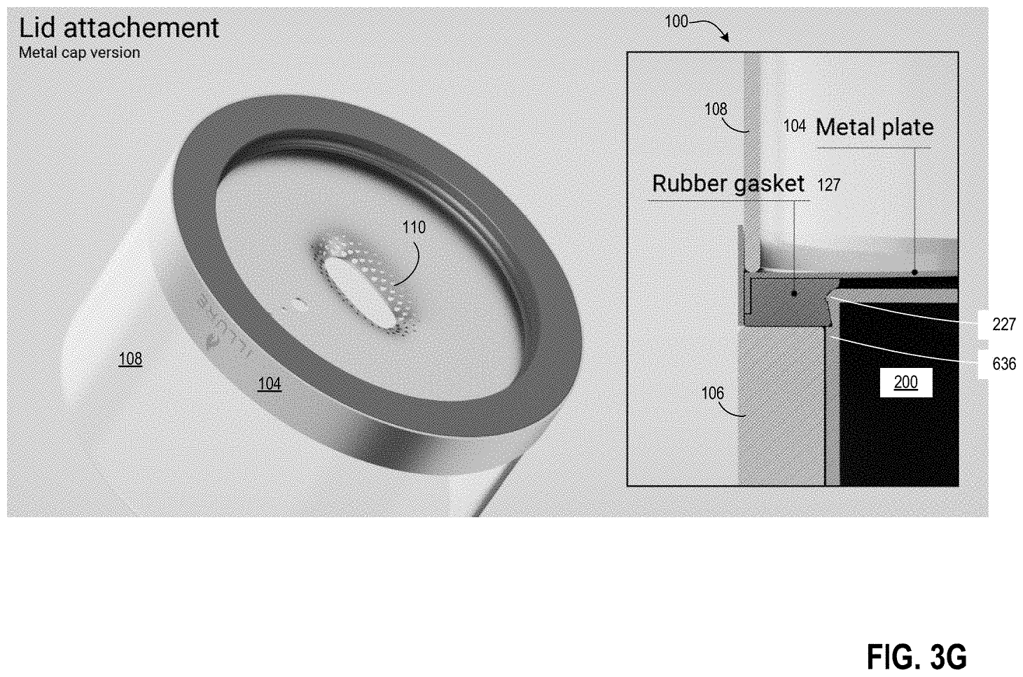

FIGS. 3A-B depicts the simulator with the silicon lid attachment 204 and the metal plate chimney 210 for the example candle simulator of FIG. 2B. As shown in FIG. 3A, the silicon cap 212 can attach to the sleeve 206. The chimney 210 can be configured to the silicon cap 212. In other implementations, the chimney 210 can be configured to the module 200 (e.g., fastened/bolted). The silicon cap 212 can have an opening configured to encircle or enclose the chimney 210 that is configured to the module 200.

As shown in FIG. 3B, once the silicon cap 212 is removed, a top surface 215 of the module 200 can be exposed. The metal plate chimney 210 can be retained to the top surface 215 of the module 200. For example, the chimney 210 can be bolted or fastened to the top surface 215 using one or more screws, bolts, or fasteners. Bolting the chimney 210 to the top surface 215 of the module 200 can be advantageous to configure the module 200 to different housings. For example, the chimney 210 can be removed so that the module 200 can be fitted into the simulator 100 having the lid attachment 104. Bolting the chimney 210 to the module 200 can be advantageous to configure the module in the simulator with the silicon lid attachment 204.

In other implementations, the chimney 210 can also be configured to the top surface 215 during manufacturing of the module 200. In other words, the chimney 210 may not be removable from the top surface 215. This can be advantageous where the module 200 is configured to fit within the simulator with the silicon lid attachment 204.

FIG. 3C depicts components of the candle simulator with the silicon lid attachment 204 of FIG. 2B. In some implementations, one or more components of the simulator 204 can be configured or attached together (e.g., at manufacturing). For example, all components 212 and 206 can be attached together except for the glass lid 208. As shown, the silicon cap 212 can have a lip or protruding edge 214. The edge 214 can secure around a top of the sleeve 206 to prevent water from spilling when the water cartridge within the module 200 is filled.

The silicon cap 212 can have an opening 217 for receiving or fitting around the metal plate chimney 210. Once the silicon cap 212 is removed or detached from the sleeve 206, the top surface 215 of the module 200 can be exposed. The top surface 215 can include the opening 220, which can be connected to the water cartridge and used to fill the water cartridge with water, as well as the chimney 210, and openings 218A-N. The openings 218A-N can include an IR window for an infrared receiver sensor and one or more LED spots. For example, the IR window can provide a viewing of light that indicates whether a remote control is in communication (e.g., wireless and/or BLUETOOTH) with the module 200. For example, the remote control can be connected to the module 200 so that the user can adjust one or more features or characteristics of a faux flame. The IR window can also include a sensor (e.g., infrared receiver sensor) or other type of receiver that can be used to connect the remote control to the module 200. In some implementations, the LED spots can indicate a battery of the module 200. For example, if the module 200 uses a rechargeable battery, once the rechargeable battery needs to be charged and/or replaced, the LED spots can change colors. A green color can be associated with full charge, an orange color can be associated with half charge, and a red color can be associated with a low charge.

As depicted, the silicon cap 212 can have an opening 219 that corresponds to a position and size of one or more of the openings 218A-N in the top surface 215 of the module 200. In the example of FIG. 3C, the opening 219 is configured to match the position and size of the IR window. Light from the LED spots (e.g., other openings 218A-N) can be configured bright enough to shine through the material of the silicon cap 212. As a result, the silicon cap 212 may not require additional openings like the opening 219 that match or correspond to all the openings 218A-N.

FIG. 3D depicts removing components of the candle simulator with the silicon lid attachment 204 of FIG. 2B. As shown, the silicon cap 212 can be snapped onto or sealed onto the top of the sleeve 206. The glass lid 208 can also be placed over the silicon cap 212 around edges of the cap 212 such that the lid 208 is flush with the edge of the sleeve 206. In other words, the lid 208 may not extend or protrude out from sides of the sleeve 206. This can provide for an aesthetic and appealing appearance.

The glass lid 208 can be removed. The silicon cap 212 can then be snapped or peeled off of the top of the sleeve 206. The cap 212 can be peeled off using the edge 214, which can be configured to secure around a top edge of the module 200 once it is placed inside the sleeve 206. Peeling off the silicon cap 212 can reveal a first portion 228 of an underside of the silicon cap 212 as well as the module 200. As described throughout this disclosure, once the silicon cap 212 is removed, the user can access components of the module 200, such as the water cartridge.

FIG. 3E depicts a bottom view of the silicon cap 212 described herein. In this example, the underside of the silicon cap 212 has the first portion 228 and a second portion 226. In some implementations, both portions 226 and 228 can be lifted or bent to peel the cap 212 off of the housing 206. In other implementations, the first portion 228 can be lifted or bent to peel the cap 212 off of the housing 206, as depicted in FIG. 3D. Moreover, the cap 212 includes the opening 217, configured to receive the metal chimney 210. The cap 212 can optionally include openings 219A-N that are positioned and a same size as the openings 218A-N in the module 200. For example, one or more of the openings 219A-N can be positioned and aligned with the LED spots and/or the IR window described herein. As shown in other implementations, the silicon cap 212 can have one opening 219 configured to align with the IR window of the module 200. Lights emitted from the LED spots on the module 200 can then filter through the material of the silicon cap 212.

The silicon cap 212 has the lip or undercut 214, which can be configured to position on or encircle a protrusion of a top surface of the module 200. Once the silicon cap 212 is positioned over the module 200, the lip 214 can seal contents of the module 200 within such that the contents do not spill out from the module 200 (e.g., refer to FIG. 3F). For example, if the water cartridge is filled with water, when the silicon cap 212 is positioned over the module 200, even if the module 200 is tipped over, the water may not spill out. The cap 212's lip 214 can create a sealed barrier.

FIG. 3F depicts attachment of the silicon cap 212 to the candle simulator 204. As shown, the module 200 has a housing 636. The housing 636 has a protrusion 227 at the top surface of the module 200. The silicon cap 212 can be positioned over the protrusion 227 and the lip 214 can seal around the protrusion 227 to create a barrier. This barrier can prevent water from spilling out of the water cartridge of the module 200, as described herein.

FIG. 3G depicts attachment of the lid attachment 104 to the candle simulator 100. A rubber gasket 127 can be positioned between the metal lid 104 and an edge or side of the protrusion 227 of the module 200. This configuration can be advantageous to prevent water from spilling out of the water cartridge within the housing 636 of the module 200. In other words, the metal plate 104 and the rubber gasket 127 can form a sealed barrier similar to the barrier of the silicon cap 212 described throughout this disclosure (e.g., refer to FIG. 3F).

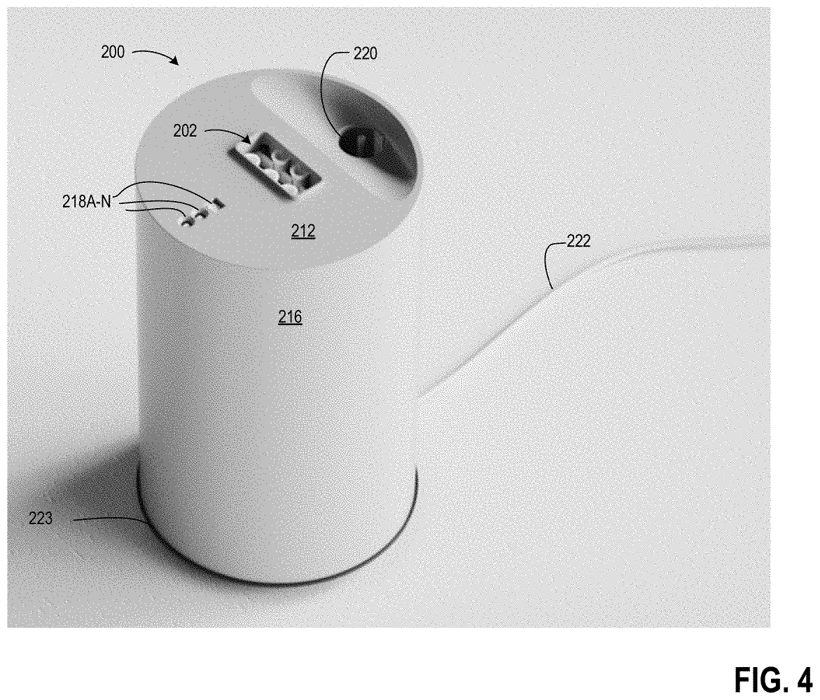

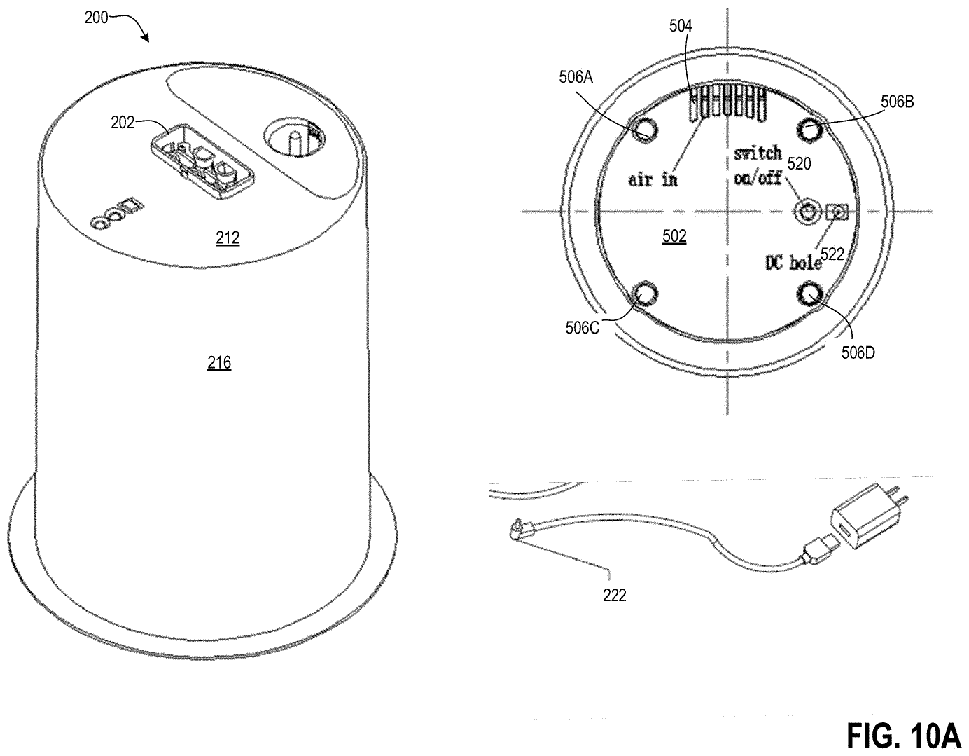

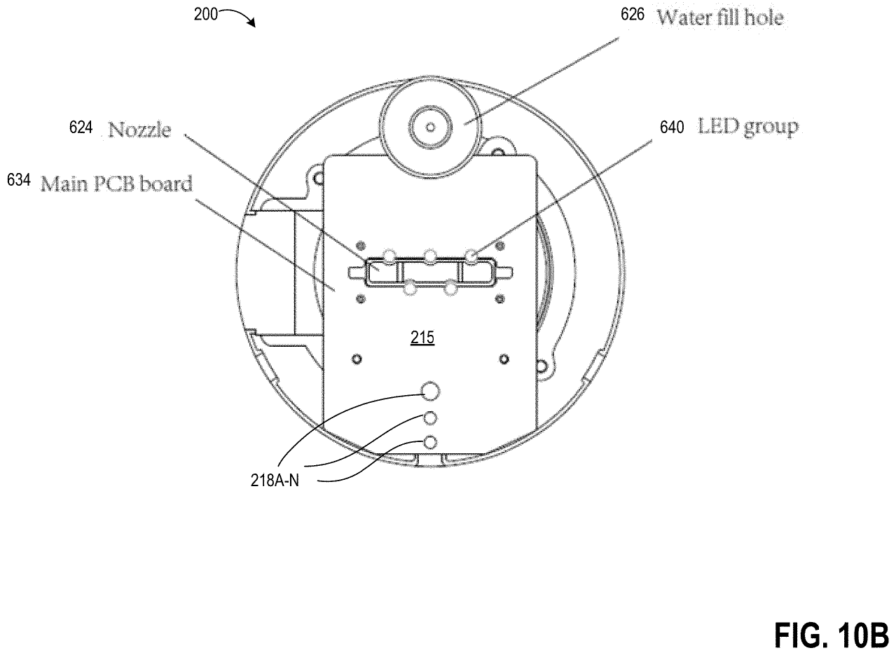

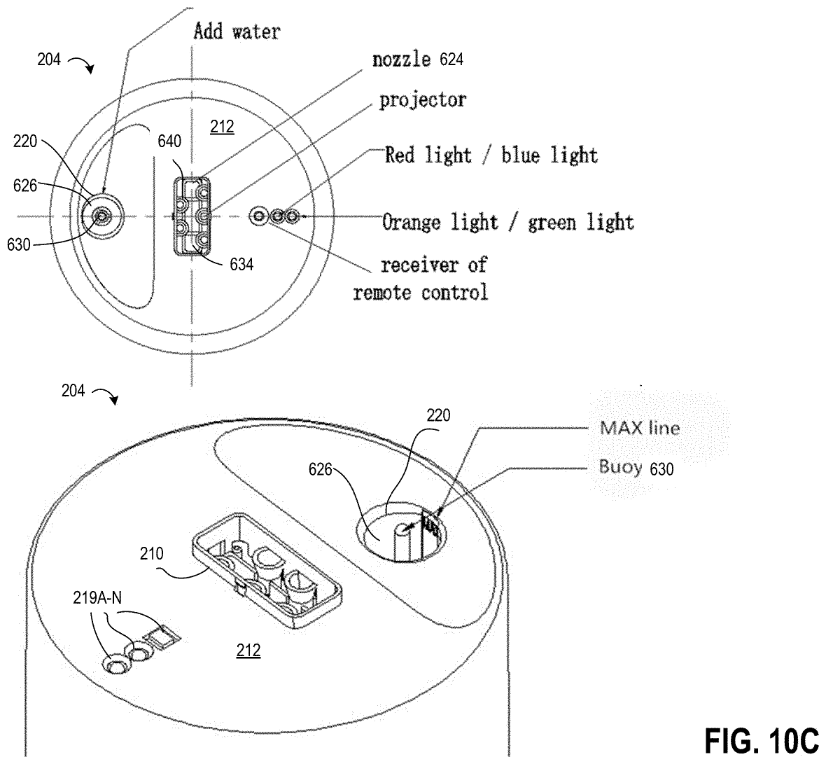

FIG. 4 depicts an example candle simulator module 200 of FIG. 2A. FIGS. 10A-C also depict the candle simulator module 200. Referring to the FIGS. 4 and 10A-C, the module 200 can include a cable 222 for charging the module 200. The cable 222 can be a USB or other cable that can be used to charge an internal power source of the module 200. Therefore, the module 200 can be operated using the internal power source. The module 200 can also be operated while the cable 222 is plugged in and charging the module 200. The module 200 can also include a sleeve 216 having a flange 223 to support various materials of the sleeves 106 and/or 206 described throughout this disclosure. Moreover, the opening 220 can take up a larger area or portion of the top surface 212 to make it easier for the user to fill the water cartridge with water. Moreover, water can be funneled through the opening 220 more easily since the opening 220 is larger and inclined downwards (e.g., sloped) towards the water cartridge within the module 200.

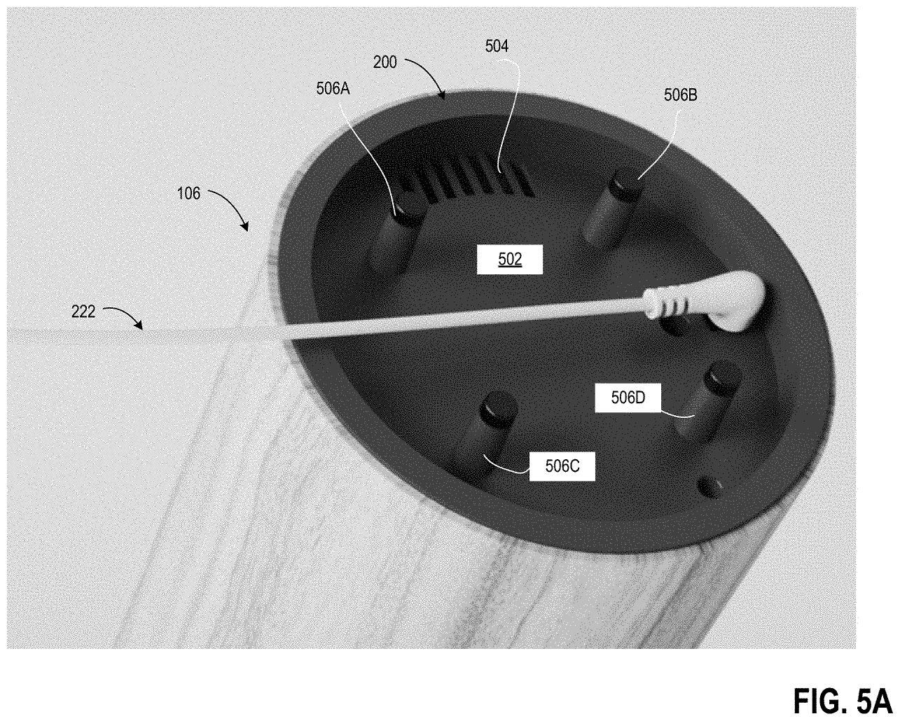

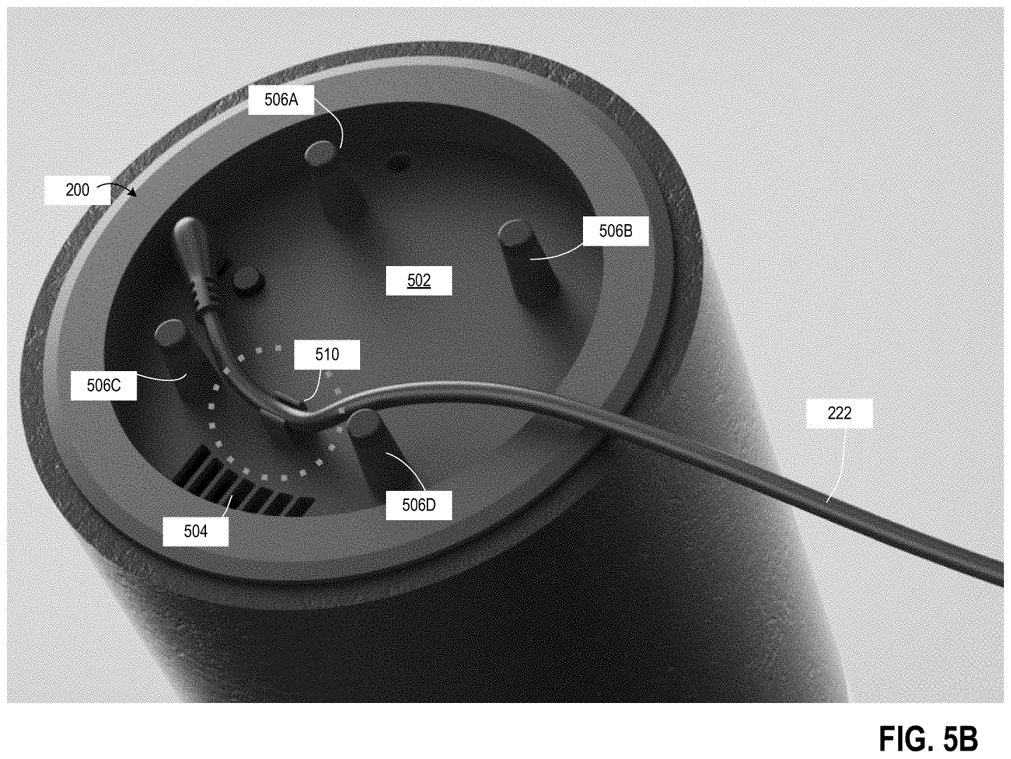

FIG. 5A depicts a bottom view of the candle simulator module 200 described herein. The module 200 can be positioned within the sleeve 106. In other implementations, the module 200 can be positioned within one or more other sleeves (e.g., the sleeve 206). As shown in FIGS. 5A and 10A, the module 200 includes a bottom cap 502. In some implementations, the bottom cap 502 can be made of a metal material. The bottom cap 502 can also have a flange (e.g., the flange 223 in FIG. 4) that is configured to support the outer sleeve 106. In some implementations, the flange can be a 2 mm surface. The bottom cap 502 also includes legs 506A-D. The legs 506A-D can be rubber. The legs 506A-D can be used to elevate the module 200 above a surface such that air can flow in through the bottom cap 502 via air openings 504. The air openings 504 can be hidden from view on the bottom cap 502 to provide a more decorative and aesthetically appealing module 200. The promotion of air flow described herein can be advantageous to generate mist and a faux flame, as described further below. The module 200 further includes the cable 222. The cable 222 can be removably attached to a port (e.g., DC Port) in the bottom cap 502. For example, once an internal battery source of the module 200 is charged, the cable 222 can be removed such that aesthetic appeal of the candle simulator may not be diminished. As another example, the candle simulator can be used while the cable 222 is attached to the module 200 and providing power to one or more components of the module 200. Since the module 200 can be elevated off the surface by the legs 506A-D, the cable 222 may not cause the module 200 to be off-balance or otherwise not leveled. The cable 222 can be positioned in a space between the surface that the module 200 is resting on and the flange or portion of the bottom cap 502 that is flush with the outer sleeve 106.

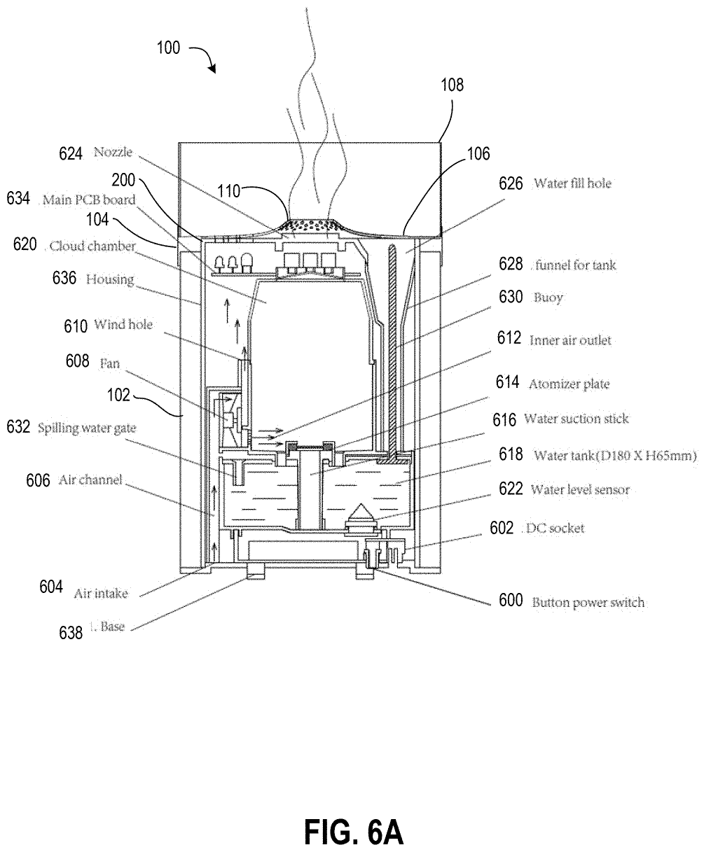

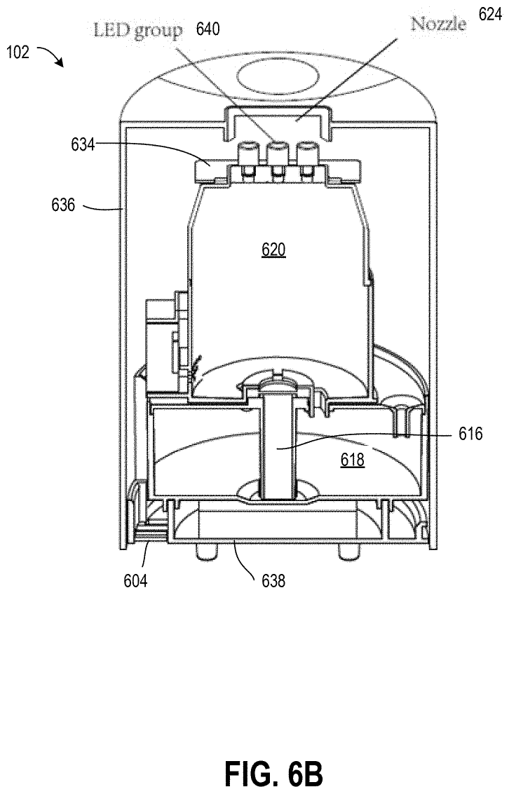

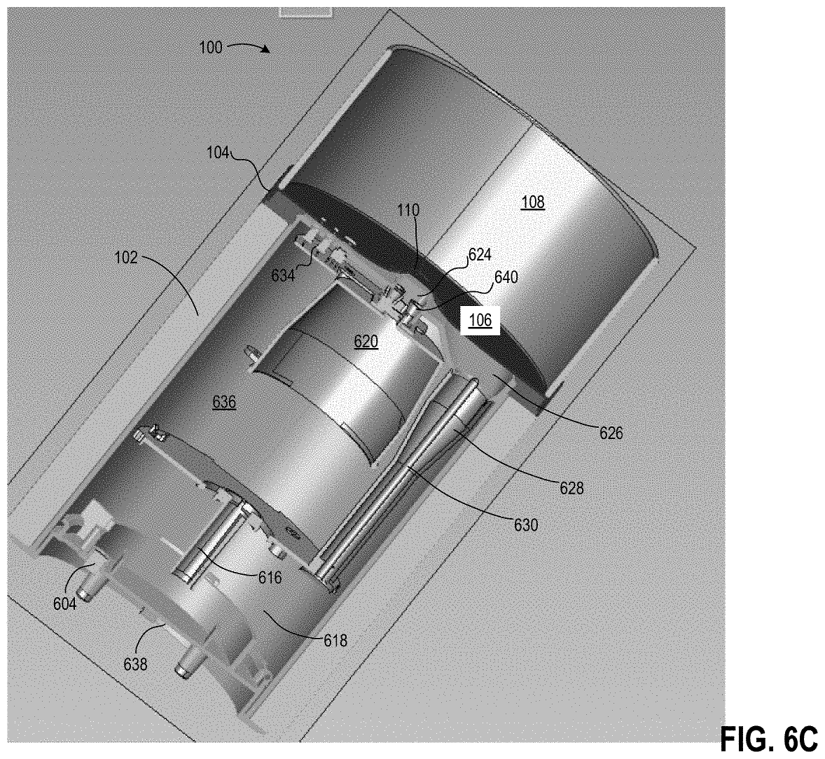

As shown in FIG. 10A, the bottom cap 502 can also include a switch 520 and a DC port 522 (e.g., refer to FIGS. 6A-C). The switch 520 can be a button or other switch that can be turned on and off to actuate the module 200 and components therein. The DC port 522 can be configured to receive the cable 222 to power the module 200 and/or charge an internal power source of the module 200.

FIG. 5B depicts a bottom view of the candle simulator module 200 with cable management 510. In comparison to the module 200 of FIG. 5A, the module 200 of FIG. 5B includes the cable management 510. The cable management 510 can be a hook, fastener, clip, or other similar means configured to retain a portion of the cable 222 to the bottom cap 502. As a result, the cable 222 may not move around and may instead be routed in a desired configuration to maintain aesthetic appeal. The cable 222 can also be routed using the management 510 away from the air openings 504 to ensure that an optimal amount of air can flow through the openings 504 and into the module 200. The cable 222 can also be routed using the management 510 in such a way to avoid the cable 222 from accidentally being positioned under one of the legs 506A-D when the module 200 is standing and/or being used to generate a faux flame.

FIGS. 6A-B are schematic cutout side views of an example candle simulator 100.

FIG. 6C is a cutout side view of components of the candle simulator 100 having the lid attachment 104. Referring to FIGS. 6A-C, the module 200 has one or more components. A power switch 600 is positioned on a base 638 (e.g., bottom cap 502 in FIGS. 5A-B). The switch 600 can also be a button or other actuator to turn on one or more components of the module 200 to simulate a faux flame. The base 638 can also include a DC socket 602 configured to receive the cable 222, as described herein. The base 638 can also include an air intake 604 (e.g., air openings 504 in FIGS. 5A-B). The air intake 604 can be configured to suck in or bring in ambient air from an external environment. Air can flow through an air channel 606 and can be dispersed through a cloud chamber 620 and a wind hole 610 via a fan 608. The air can flow into the cloud chamber 620 via an inner air outlet 612 and by the fan 608.

The user can fill a water tank 618 (e.g., water cartridge) by pouring water through a water fill hole 626 (e.g., the opening 220 described throughout this disclosure). In some implementations, the tank 618 can be a D180×H65 mm. One or more other tank configurations or tanks can be incorporated into the module 200. The water can filter through a funnel 628, which siphons the water into the tank 618. A buoy 630 can be positioned within the funnel 628 as a gauge for the user to determine how much water is in the tank 618. The buoy 630 can have a flat end that floats inside the tank 618 that can seal off an opening in the tank 618 where the water filters in from the funnel 628. A water level sensor 622 can also be configured within the tank 618 to determine a fill level of the tank 618. When the tank 618 is filled, the buoy 630 can be pushed up against the opening in the tank 618 where the water filters in from the funnel 628. This can cause the tank 618 to be sealed off from receiving additional water. When the buoy 630 pushes up against the opening in the tank 618, the user can see the buoy 630 protruding from the water fill hold 626, which can indicate that the tank 618 is full and no more water should be added. In some implementations, when the water level sensor 622 detects that the tank 618 is full, the sensor 622 can communicate a signal to one of the LED spots positioned on a main PCB board 634. The LED spot can be illuminated a color indicative of a water fill level. For example, the LED spot can glow green when the tank 618 is full. The LED spot can glow red when the tank 618 is empty.

The tank 618 can include a spilling water gate 632. The gate 632 can be configured to prevent water inside the tank 618 from spilling out into the air chamber 606 or other components of the module 200. The tank 618 can also include a water suction stick 616. The water suction stick 616 can be configured to suction or pull water from the tank 618 and up into the cloud chamber 620. An atomizer plate 614 can be positioned at a top of the water suction stick 616. The atomizer pate 614 can be configured to generate mist in the cloud chamber 620 using the air brought in via the air channel 606 and the water brought in via the water suction stick 616. The generated mist can be propagated around in the cloud chamber 620, through the wind hole 610, and out through a nozzle 624. As described above, the fan 608 can create a flow path for the mist such that the mist propagates out of the nozzle 624.

Still referring to the FIGS. 6A-B, the main PCB board 634 can include the LED spots and/or the IR sensor described above. The board 634 can also be configured to an LED group 640. The LED group 640 can include one or more LED lights that are directed up towards the nozzle 624. The LED group 640 can also be positioned such that each of the lights in the LED group 640 illuminate the generated mist at different angles along a flow path for the mist from the cloud chamber 620, through the wind hole 610, and out through the nozzle 624. For example, the lights in the LED group 640 can be positioned at different angles (e.g., one can be directed towards a right side of the nozzle 624, another can be directed towards a left side of the nozzle 624, and another can be directed at a top opening of the nozzle 624). As the generated mist is propagated out through the nozzle 624, the mist and light from the LED group 640 can be dispersed through apertures, holes, or other openings in the chimney 110. This can provide a more realistic looking flame than if the generated mist is propagated through a central opening. The various differently shaped, positioned, and/or sized apertures in the chimney 110 can be advantageous to form the flame and/or create differently designed or shaped flames.

In some implementations, the module 200 can also emit a fragrance or other aroma or scent. For example, a fragrance can be added to water that is filtered through the funnel 628 into the tank 618. When mist is generated, the fragrance can then be emitted or dispersed along with the mist through the nozzle 624. As another example, the module 200 can be configured to provide for separate water nebulization and fragrance nebulization. For example, a twist and lock cartridge can be used to contain a fragrance. Mist can be generated using the water in the tank 618 and another mist can be generated using the fragrance in the cartridge. The two mists can be combined and expelled through the nozzle 624. The two mists can also be separately expelled through the nozzle 624 or two different openings. For example, the fragrance can be expelled through openings in the base 638. The fragrance can also be expelled through an opening in the top surface 106 that is different from the nozzle 624 and/or the chimney 110. As yet another example, the top surface 106 can have a spout or opening for the fragrance that is separate or different than the water fill hole 626. The fragrance can flow into a tank that is separate or different than the water tank 618. Moreover, the fragrance can be atomized using an atomizer plate that is separate or different than the atomizer plate 614. Once the fragrance and water are atomized separately, they can be mixed together in the cloud chamber 620 and expelled out through the nozzle 624. As mentioned, the atomized fragrance and water can also be separately moved through the module 200 and out through different openings. A fan that is separate or different than the fan 608 can be used to direct the fragrance through openings in the module 200.

In other implementations, the module 200 can generate heat from the faux flame. Therefore, the module 200 can include a digital temperature control. The module 200 can have a thermostat (e.g., temperature sensor) and a display (e.g., LCD, LED, OLED, etc.) that can be configured to display a temperature of the generated flame. The user can adjust an amount of heat that is generated by the flame based on viewing the displayed temperature. In some examples, the temperature can be displayed on a remote control that is in communication with the module 200. The user can then adjust the temperature of the flame using the remote control. In other examples, the temperature can be displayed on a mobile device (e.g., cellphone, smart phone, tablet, laptop, computer, etc.) via a mobile application from which the user can also adjust or moderate temperature of the flame.

In yet other implementations, the faux flame can be adjusted based on fan speed. Adjusting fan speed can cause the flame to be tamed (e.g., smaller), maxed out (e.g., larger), and/or any strength in between. For example, the flame can be increased in height by increasing speed of the fan 608. The flame can also be made narrower by increasing speed of the fan 608. As another example, the flame can be decreased in height or made wider by decreasing speed of the fan 608. In some implementations, adjusting fan speed can also effect an amount of heat generated by the flame. The user can adjust fan speed using the remote control or the mobile application on the mobile device, as described above.

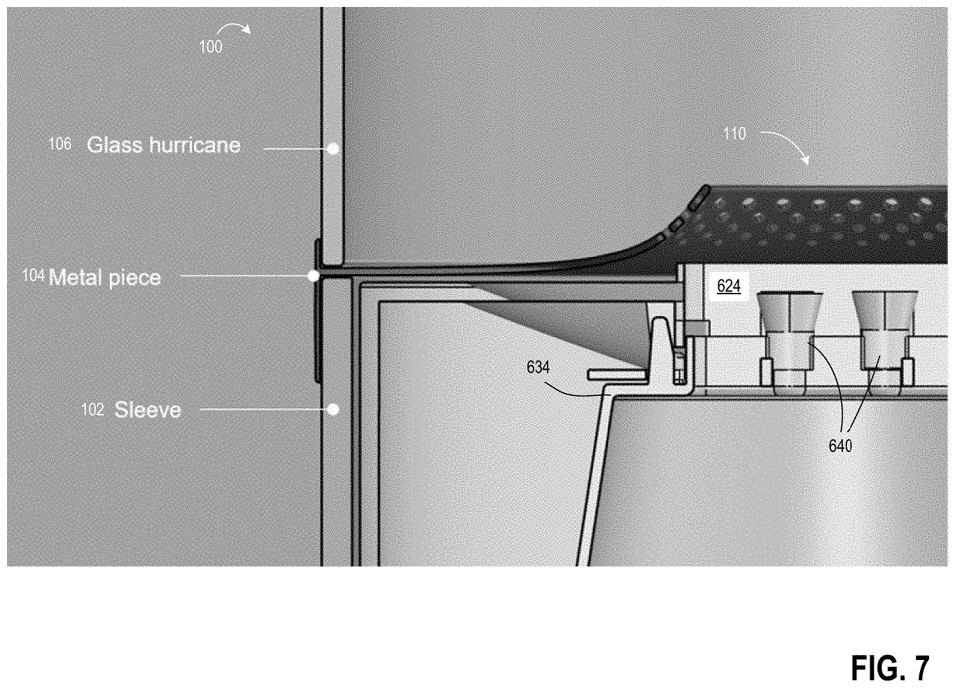

FIG. 7 is a schematic cutout side view of the candle simulator 100 having the lid attachment 104. The lid attachment 104 has the chimney 110 configured with various apertures of differing sizes. When generated mist is propagated out through the nozzle 624 and illuminated by the LED group 640, the mist can propagate through the apertures in the chimney 110 to look like a realistic flame.

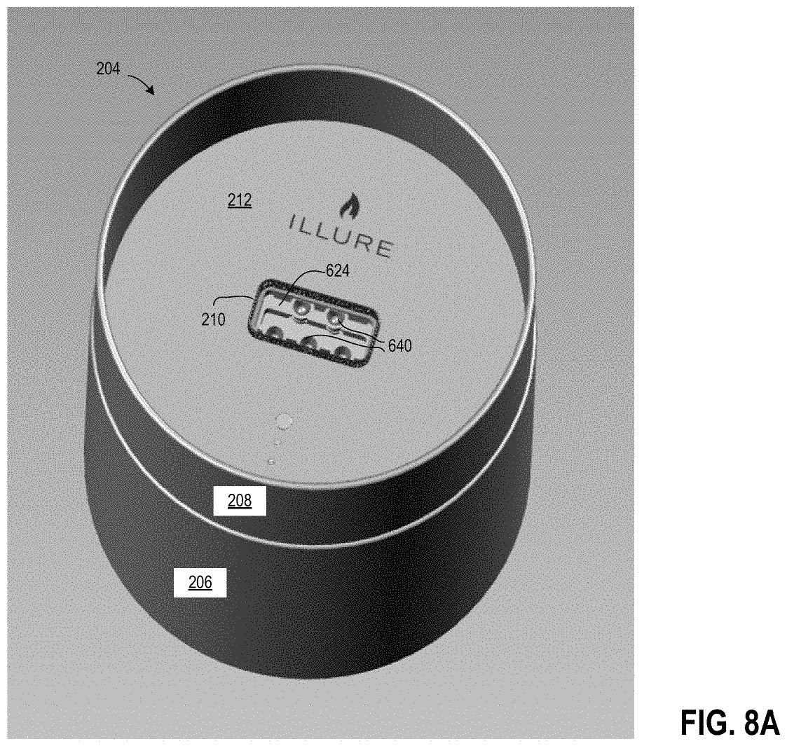

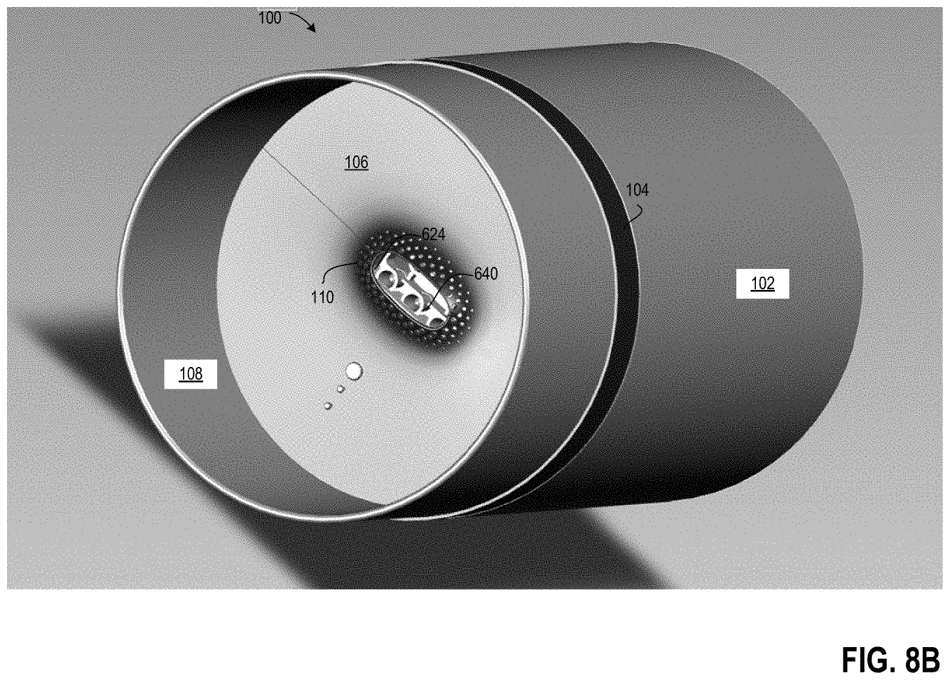

FIG. 8A is a top down view of the candle simulator having the silicon lid attachment 204. Generated mist can be propagated through the nozzle 624, up through the wide opening of the metal plate chimney 210, and also out through sides of the metal plate chimney 210. As depicted and described throughout this disclosure, sides of the chimney 210 can have apertures of varying sizes and shapes to provide for a more realistic-looking flame. In this example candle simulator with the silicon lid attachment 204, the LED group 640 has five LED lights. In other example candle simulators (e.g., refer to FIG. 6A, FIG. 8B), the LED group 640 can have three LED lights. In yet other example candle simulators, the LED group 640 can have one or more additional or fewer LED lights.

FIG. 8B is a top down view of the candle simulator 100 having the lid attachment 104. Generated mist can be propagated through the nozzle 624, up through the top opening of the chimney 110, and also out through sides of the chimney 110. As depicted and described throughout this disclosure, sides of the chimney 110 can have apertures of varying sizes and shapes to provide for a more realistic-looking flame. In this example candle simulator 100, the LED group 640 has three LED lights.

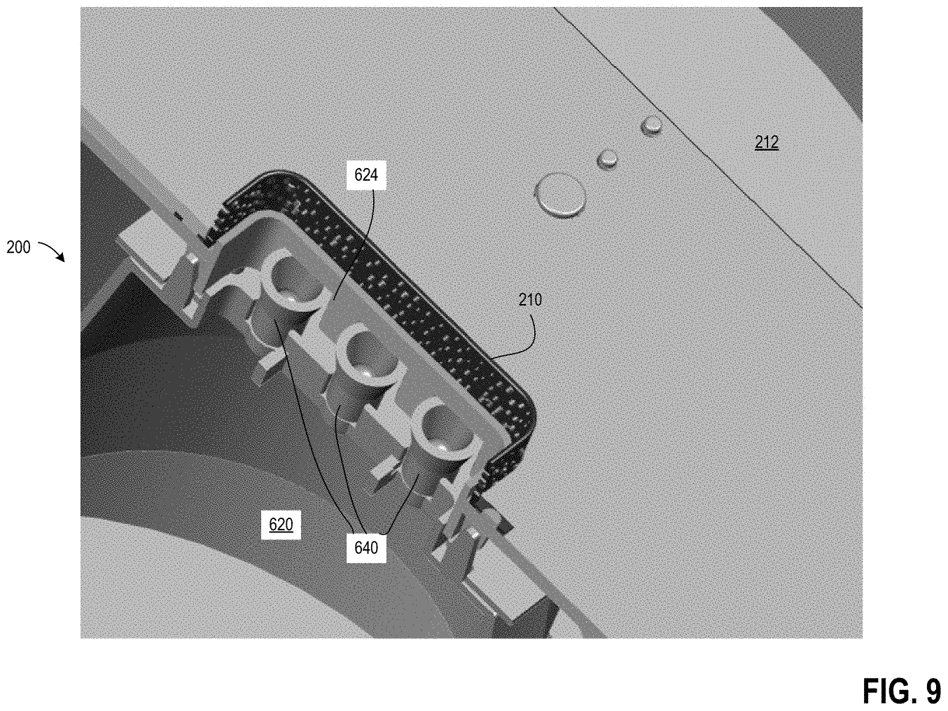

FIG. 9 is an exploded top down view of the metal plate chimney 210 and components of the candle simulator module 200. In this example, the LED group 640 has three LED lights. the LED lights of the group 640 are directed up towards the wide opening in the metal plate chimney 210. Generated mist can flow from the cloud chamber 620, around the LED group 640, through the nozzle 624, and out through the wide opening and apertures of the chimney 210. The LED lights of the group 640 can generate light of different colors, brightness, and/or intensity along the flow path of the mist, thereby illuminating the mist to create a realistic faux flame.

FIGS. 10A-B depict the candle simulator module 200. FIG. 10C depicts a top view of the candle simulator having the silicon lid attachment 204. As described in reference to the previous FIGS and as shown in FIGS. 10A-C, water can be added to the water tank (e.g., the tank 618 in FIGS. 6A-C) through the opening 220, down the water fill hole 626. The buoy 630 can raise closer to the top of the hole 626 or the opening 220 to indicate a maximum fill of the tank. For example, a maximum line can be drawn along a side of the hole 626. Then the top of the buoy 630 raises to the maximum line, the user can see that the tank is filled with water and no more water should be added.

In the example of FIGS. 10A-C, the LED group has five LED lights that can shine light through the nozzle 624 to illuminate mist that propagates out through the nozzle 624 and the metal plate chimney 210. The silicon cap 212 can also have openings 219A-N through which LED lights from the IR window and/or LED spots 218A-N of the module 200 can be outputted or displayed.

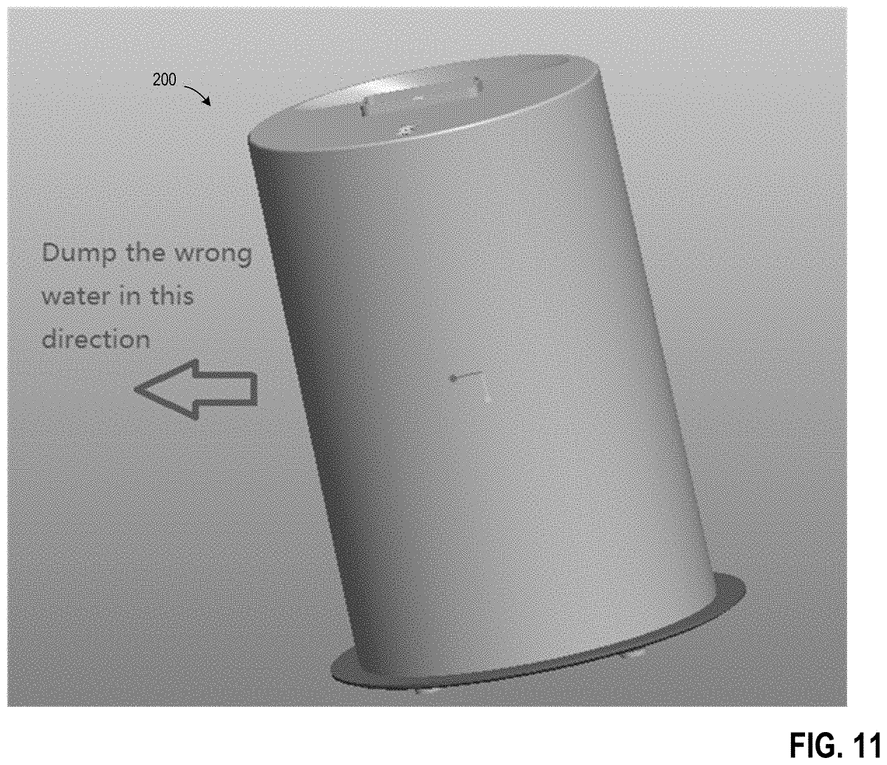

FIG. 11 depicts the candle simulator module 200 when tipped at an angle. As described throughout this disclosure (e.g., refer to FIGS. 6A-C), the module 200 can be sealed in such a way that water from the water tank may not spill out of the module 200. Therefore, the module 200 can be tipped in one or more different directions and the water can remain within the tank of the module 200.

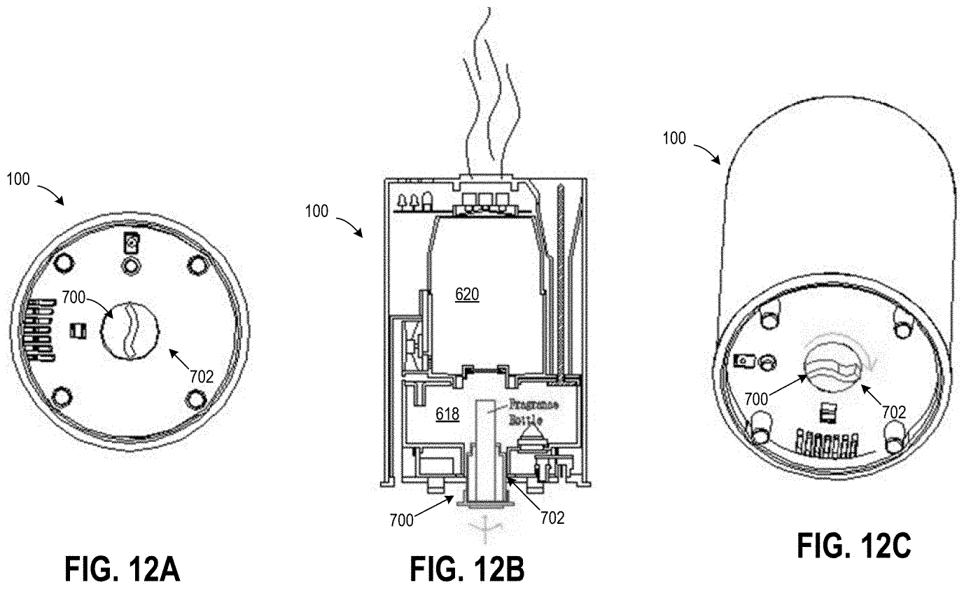

FIGS. 12A-C depict an example candle simulator 100 with an insertable fragrance bottle 700 that can add fragrance to the water tank 618 and simulated flame. FIG. 12A is a bottom view, FIG. 12B is a side view, and FIG. 12C is a perspective view of the example candle simulator 100 with insertable fragrance bottle 700. The candle simulator 100 that is depicted can include an opening 702 in a bottom surface that is configured to receive and retain the fragrance bottle 700 in fluid communication with the water tank 618. For example, the opening 702 can include a threaded portion that mates with an a opposite threaded portion on the fragrance bottle 700 to form a fluid seal (retaining fluid within the water chamber 618). The fragrance bottle 700, for example, can be twisted and locked into place via the opening 702 in the example candle simulator 100. The fragrance bottle 700 can include one or more apertures or water permeable surfaces/components that permit for fragrances (e.g., fluids, objects, material) contained in the bottle 700 to be dissolved in and/or to otherwise combine with the fluid in the water chamber 618, which can cause for the simulated flame to include fragrance. The fragrance bottle 700 can be removable and refillable, and/or can be disposable and replaceable with other bottles 700. For example, the bottle 700 can be a consumable cartridge that is purchased in different fragrances and intensities. In another example, the bottle 700 can be refillable by the user and can permit the user to add different fluids and/or fragrant objects (e.g., fruits, flowers, spices) into the bottle 700 to infuse fragrances into the water chamber 618. Other configurations for adding fragrance are also possible, such as having a separate fragrance atomizer that is included in the module.

The disclosed technology can be considered an electromechanical domestic appliance nesi, with a self-contained electric motor, such as in Harmonized Tariff Schedule (HTS) Code 8509.80.50.

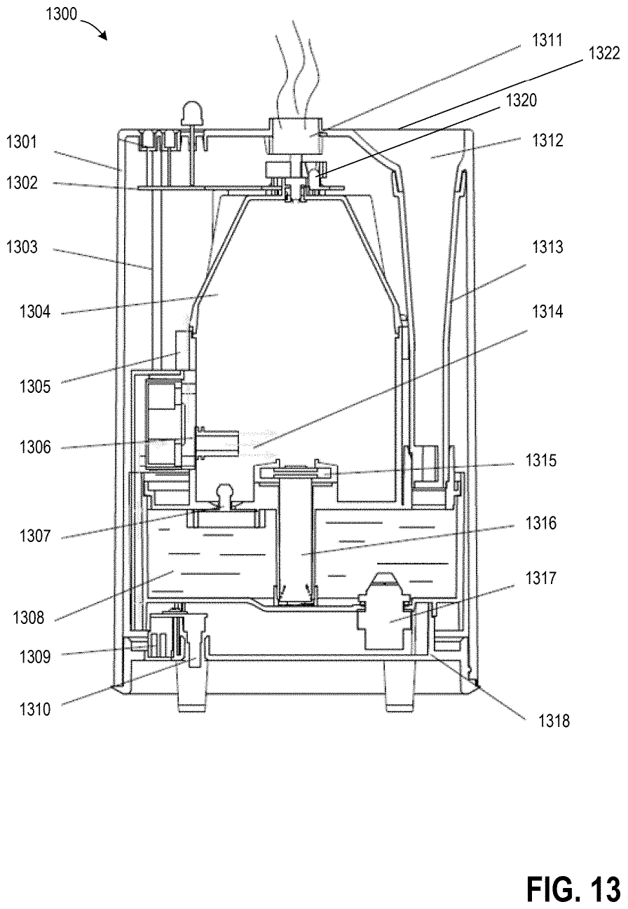

FIG. 13 is a schematic cutout side view of an example candle simulator 1300. The candle simulator 1300 can operate and function similarly to the candle simulators described throughout this disclosure. The candle simulator 1300 can include a housing 1301, a main PCB board 1302, an air pipe 1303, a cloud chamber 1304, an air outlet 1305, a fan 1306, a valve 1307 (e.g., silicone), a water tank or other fluids tank 1308, a DC power socket 1309, a button switch 1310, a nozzle 1311, a water fill hole 1312, a water funnel 1313, an inner chamber outlet 1314, an atomizer 1315, a water suction stick 1316, a water level sensor 1317, and a bottom holder 1318. The nozzle 1311 can be sized such that a light shining through the nozzle 1311 can appear brighter, thereby improving a flame effect of the candle simulator 1300. The nozzle 1311, which may be considered a chimney by itself and/or in combination with a portion of a lid, and can focus the flow of gas from the cloud chamber so that it has the appearance of a flame (e.g., flame effect), particularly when illuminated by one or more lights 1320 that are positioned adjacent and/or below the nozzle. The nozzle 1311 can be configured to extend above a top surface 1322 of the housing 1301 so as to mate with a corresponding opening in the lid (not depicted). The nozzle 1311 can, for example, extend above the top surface 1322 by a sufficient amount so that it is flush with a top surface of the lid, extends above the top surface of the lid, and/or is positioned below a top surface of the lid when the lid is positioned on the housing 1301. Moreover, the fan 1306 can be adjusted to one or more different speeds. A higher speed, for example, can result in an improved, realistic flame effect while a slower speed can result in a smaller flame effect.

Unlike the candle simulators previously described in this disclosure, the candle simulator 1300 includes the valve 1307 instead of a buoy that passes through the water fill hold 1312 (refer to the buoy 630 in FIGS. 6A-B). The valve 1307 can be configured to operate similarly to the buoy described herein. For example, the valve 1307 can be configured to prevent water or other fluids, including but not limited to water vapor, from transferring between the water tank 1308 and the cloud chamber 1304. Thus, the valve 1307 can fluidically separate the water tank 1308 from the cloud chamber 1304. The valve 1307 can be a one way valve, in some implementations. As a result, water or other fluids may only flow down from the cloud chamber 1304 into the water tank 1308.

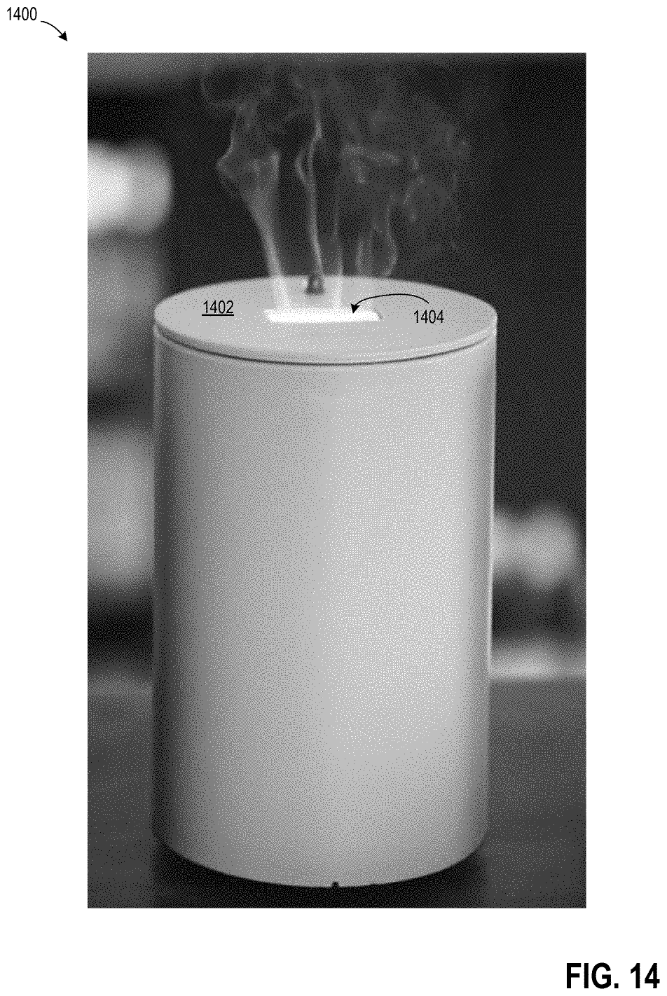

FIG. 14 depicts a front view of an example candle simulator 1400 without a transparent lid. The candle simulator 1400 operates and functions similarly to the candle simulators described throughout this disclosure. For example, the candle simulator 1400 can be the candle simulator 1300 or any other candle simulators described herein. However, unlike the candle simulator 100, a chimney (such as the chimney 110 described in FIGS. 1A-C) can be embedded inside the candle simulator 1400 instead of protruding above a top surface 1402 of the candle simulator 1400. The chimney (not depicted) can therefore be aligned with an opening 1404 in the top surface 1402 of the candle simulator 1400 through which a realistic faux flame is emitted. Moreover, the candle simulator 1400 may not include a transparent lid, such as the transparent lid 108 of the candle simulator 100 (refer to the transparent lid 108 described in FIGS. 1A-D).

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

What is claimed is:

1. An apparatus for providing a simulated flame, the apparatus comprising:
a base housing;
a flow generator contained within the base housing that is configured to generate a flow of atomized fluid;
a lid positioned on top of the base housing, the lid defining a main opening through which the flow of atomized fluid is configured to be emitted;
a chimney that is positioned adjacent the main opening, the chimney extending upwards and configured to focus, at least in part, the flow of atomized fluid into a channel of atomized fluid; and
one or more light sources that are positioned near the main opening to the lid that are configured to illuminate the channel of atomized fluid to provide the simulated flame.

2. The apparatus of claim 1, wherein the chimney is attached to a top surface of the lid and that, at least partially, surrounds the opening, the chimney extending upward from the top surface of the lid and being configured to focus, at least in part, the flow of atomized fluid into the channel of atomized fluid.

3. The apparatus of claim 2, wherein one or more sidewalls of the chimney define one or more apertures that are configured to promote, at least in part, the formation of the channel of atomized fluid by the chimney.

4. The apparatus of claim 3, wherein the one or more sidewalls extend orthogonally from the top surface of the lid.

5. The apparatus of claim 3, wherein the one or more sidewalls comprise one or more curved surfaces that extend from the top surface of the lid.

6. The apparatus of claim 3, wherein the one or more sidewalls comprise one or more planar surfaces that extend from the top surface of the lid.

7. The apparatus of claim 3, wherein the one or more sidewalls taper from their attachment to the top surface of the lid to a terminal point above the top surface.

8. The apparatus of claim 1, further comprising:
a transparent lid that extends upward from a top surface of the lid, the transparent lid at least partially enclosing a volume that is configured to contain the simulated flame.

9. The apparatus of claim 8, wherein the transparent lid defines a first opening that is configured to mate with the lid and a second opening that is configured to be open to an ambient environment.

10. The apparatus of claim 1, wherein the chimney is part of the base housing.

11. The apparatus of claim 10, wherein the chimney extends through the main opening to the lid.

12. The apparatus of claim 1, further comprising:
a cloud chamber embedded inside the base housing and fluidically connected to the main opening to the lid and the flow generator;
a liquid chamber embedded inside the base housing and positioned beneath a portion of the cloud chamber; and
a valve positioned inside the base housing to fluidically separate the cloud chamber from the liquid chamber, wherein the valve is configured to prevent liquid from flowing from the liquid chamber into the cloud chamber.

13. The apparatus of claim 12, wherein the valve is a one-way valve.

14. The apparatus of claim 12, wherein the valve is a silicone valve.

15. The apparatus of claim 12, further comprising a fan embedded inside the base housing, wherein the fan is configured to circulate the flow of atomized fluid from the flow generator through the cloud chamber and out through the main opening to the lid to provide the simulated flame.

16. The apparatus of claim 15, wherein a speed of the fan is adjustable so as to change an appearance of the simulated flame.

17. The apparatus of claim 16, wherein a higher fan speed increases the flow of atomized fluid to provide a stronger simulated flame and a lower fan speed decreases the flow of atomized fluid to provide a slower simulated flame.
